(19) United States
(12) Patent Application Publication
(10) Pub. No.: US 2008/0242740 A1
(43) Pub. Date: Oct. 2, 2008

(54) AROMA COMPOSITIONS OF ALKAMIDES WITH HESPERETIN AND/OR 4-HYDROXYDIHYDROCHALCONES AND SALTS THEREOF FOR ENHANCING SWEET SENSORY IMPRESSIONS

(75) Inventors: Jakob Ley, Holzminden (DE); Kathrin Freiherr, Dassel (DE); Gerhard Krammer, Holzminden (DE); Gerald Reinders, Hoexter (DE)

Correspondence Address: CONNOLLY BOVE LODGE & HUTZ, LLP, P O BOX 2207, WILMINGTON, DE, 19899, US

(73) Assignee: SYMRISE GmbH & Co. KG, Holzminden (DE)

(21) Appl. No.: 12/058,088

(22) Filed: Mar. 28, 2008

Related U.S. Application Data
(60) Provisional application No. 60/908,730, filed on Mar. 29, 2007.

(51) Int. Cl. A23L 1/236; A61K 47/16

(52) U.S. Cl. 514/784; 426/536; 514/788

(57) ABSTRACT

The invention primarily relates to aroma compositions of (i) certain saliva-stimulating alkamides having a tingling, pungent and/or hot flavor (such as for example pellitorines, spilanthol) with (ii) hesperetin (5,7-dihydroxy-3-(3-hydroxy-4-methoxyphenyl)-chroman-4-one) or the enantiomers and/or salts thereof and/or (iii) 4-hydroxydihydrochalcones (3-(4-hydroxyphenyl)-1-phenylpropan-1-ones) and/or the salts thereof, the use thereof to enhance the sweet flavor of sweet-tasting substances or the sweet odor impression of aroma substances which give rise to a sweet odor impression, but in particular to enhance the sweet initial flavor or odor (initial sweetness). The invention thus relates to the use of said aroma compositions as general sweetness enhancers and enhancers of initial sweetness. The invention further relates to certain preparations which contain an effective amount of said aroma compositions of (i) alkamides (such as in particular pellitorines, spilanthol) with (ii) hesperetin or the enantiomers and/or salts thereof and/or (iii) 4-hydroxydihydrochalcones and/or the salts thereof and methods for enhancing the sweet flavor or initial sweetness of a sweet-tasting substance or the sweet odor impression or initial sweetness of an aroma substance which gives rise to a sweet odor impression. The invention also relates to the use of certain alkamides (i) to enhance the initial sweetness of a preparation comprising (ii) hesperetin or the enantiomers and/or salts thereof and/or (iii) 4-hydroxydihydrochalcones and/or the salts thereof and (b) sweet-tasting substances and/or (c) aroma substances which give rise to a sweet odor impression.

### RELATED APPLICATIONS

This application claims benefit to U.S. Provisional application 60/908,730 filed Mar. 29, 2007.

The invention primarily relates to aroma compositions of (i) certain saliva-stimulating alkamides having a tingling, pungent and/or hot flavor (such as for example pellitorines, spilanthol) with (ii) hesperetin (5,7-dihydroxy-3-(3-hydroxy-4-methoxyphenyl)-chroman-4-one) or the enantiomers and/or salts thereof and/or (iii) 4-hydroxydihydrochalcones (3-(4-hydroxyphenyl)-1-phenylpropan-1-ones) and/or the salts thereof, the use thereof to enhance the sweet flavor of sweet-tasting substances or the sweet odor impression of aroma substances which give rise to a sweet odor impression, but in particular to enhance the sweet initial flavor or odor (initial sweetness). The invention thus relates to the use of said aroma compositions as general sweetness enhancers and enhancers of initial sweetness. The invention further relates to certain preparations which contain an effective amount of said aroma compositions of (i) alkamides (such as in particular pellitorines, spilanthol) with (ii) hesperetin or the enantiomers and/or salts thereof and/or (iii) 4-hydroxydihydrochalcones and/or the salts thereof and methods for enhancing the sweet flavor or initial sweetness of a sweet-tasting substance or the sweet odor impression or initial sweetness of an aroma substance which gives rise to a sweet odor impression. The invention also relates to the use of certain alkamides (i) to enhance the initial sweetness of a preparation comprising (ii) hesperetin or the enantiomers and/or salts thereof and/or (iii) 4-hydroxydihydrochalcones and/or the salts thereof and (b) sweet-tasting substances and/or (c) aroma substances which give rise to a sweet odor impression.

Foodstuffs or products consumed for pleasure having a high sugar content (above all sucrose (=saccharose), lactose, glucose or fructose or mixtures thereof) are generally greatly preferred by consumers because of their sweetness. On the other hand, it is generally known that a high content of easily metabolizable carbohydrates causes the blood sugar level to rise sharply, leads to the formation of fat deposits and may ultimately lead to health problems such as excess weight, obesity, insulin resistance, adult-onset diabetes and associated secondary disorders. Matters are further complicated in particular by the fact that many of the aforementioned carbohydrates may additionally have a detrimental effect on dental health, since they are broken down in the oral cavity by certain types of bacteria to form lactic acid, for example, and may attack the enamel of juvenile or adult teeth (caries).

It has therefore long been an objective to reduce the sugar content of foodstuffs and/or products consumed for pleasure to the absolutely necessary level or below. A corresponding measure consists in the use of sweeteners, i.e. chemically uniform substances which themselves have no or only a very low calorific value and which at the same time give rise to a strong sweet flavor impression; the substances are generally non-cariogenic (a review may be found for example in Journal of the American Dietetic Association 2004, 104 (2), 255-275). Although the “bulk sweeteners” such as sorbitol, mannitol or other sugar alcohols are in some cases excellent sweetening agents and can also partially replace the conventional food properties of sugars, in a proportion of the population too high a consumption can lead to digestion problems due to osmosis. Owing to their low usage concentration, the non-nutritional, high-intensity sweeteners are very suitable for adding sweetness to foodstuffs, however they often exhibit flavor problems due to dissimilar intensity profiles over time in comparison to sugar (for example sucralose, stevioside, cyclamate), a bitter and/or astringent aftertaste (for example acesulfame K, saccharin) or pronounced additional aroma impressions (for example glycyrrhizic acid ammonium salt). Some of the sweeteners are not particularly heat resistant (for example thaumatin, brazzein, monellin), are not stable in all applications (for example aspartame) and are in some cases very long-lasting in their sweet effect (strong sweet aftertaste, for example saccharin).

An improvement in the flavor properties, in particular the aftertaste problem, of non-nutritional, high-intensity sweeteners can be achieved through the use of tannic acid, as described for example in WO 98/20753, or phenolic acids as in U.S. Pat. No. 3,924,017. Owing to their catechol units, however, such substances are not particularly stable in applications.

Another possibility—without the use of non-nutritional sweeteners—consists in reducing the sugar content of foodstuffs and/or products consumed for pleasure and adding organoleptically weak or imperceptible substances which enhance the sweetness directly or indirectly, as described for example in WO 2005/041684. The substances described in WO 2005/041684 are expressly of non-natural origin, however, and are thus more difficult to assess from a toxicological viewpoint than substances of natural origin, particularly when the latter occur in foodstuffs or products consumed for pleasure or derive from raw materials for the production of foodstuffs or products consumed for pleasure. EP 1 291 342 describes such substances of natural origin (pyridinium betaines); however, said substances do not influence the sweet flavor selectively but instead also influence other flavor directions such as umami or saltiness. In addition, the disclosed substances can be purified only with great effort.

In WO 2007/014879 (Symrise) the use of hesperetin is recommended as an enhancer of the sweet flavor of reduced-sugar preparations serving for nutrition or for pleasure. With the use of hesperetin, however, its low solubility, particularly in clear, aqueous applications (such as for example clear colas or lemonades), and the comparatively weakly pronounced sweetness enhancement in acidic foodstuffs and products consumed for pleasure is sometimes disadvantageous. The use of hesperetin also in combination with 4-hydroxydihydrochalcones is described in WO 2007/014879. Considerable reductions in the quantities of sugar or sweetener while maintaining identical sweetness can be achieved in this way, although the profile, particularly the initial sweetness, is occasionally not perceived as being adequate or satisfactory.

It is therefore desirable to find substances or mixtures of substances (aroma compositions) which in low concentrations effectively enhance sweet flavor impressions of sweet substances, preferably the sweet flavor impression of reduced-sugar foodstuffs and products consumed for pleasure, in particular of reduced-sugar acidic and/or clear aqueous foodstuffs and products consumed for pleasure, without negatively influencing the rest of the aroma profile. It is likewise desirable to find substances which in low concentrations effectively enhance sweet odor impressions of aroma substances which give rise to a sweet odor impression.

The primary object of the present invention was to find aroma compositions which (a) are suitable for selectively enhancing the sweet flavor of a sweet-tasting substance and/or the sweet odor impression of an aroma substance which gives rise to a sweet odor impression, preferably without negatively influencing the rest of the aroma profile, and (b) are particularly suitable for enhancing the initial sweetness of the application.

According to a first aspect of the present invention, the stated object is achieved by means of an aroma composition consisting of or containing a substance (i) and a substance (ii) or a substance (iii) or a mixture of substances (ii) and (iii), wherein:

(i) consists of one or more alkamides selected from the group consisting of:

2E,4E-decadienoic acid-N-isobutylamide (pellitorine), 2E,4Z-decadienoic acid-N-isobutylamide(cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);

(ii) is hesperetin of the formula (I)

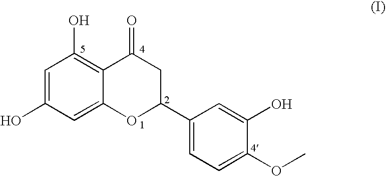

wherein the hesperetin of the formula (I) is present as the (2S)-enantiomer, (2R)-enantiomer or any desired mixture of the two enantiomers, and/or the salts thereof and

(iii) is
a 4-hydroxydihydrochalcone of the formula (II)

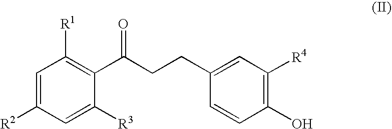

wherein

R¹, R², R³ and R⁴ in each case mutually independently denote H, OH or O-alkyl, with the proviso that at least one of the residues R¹, R² or R³ denotes OH,
a salt of such a 4-hydroxydihydrochalcone of the formula (II),
a mixture comprising or consisting of two or more different 4-hydroxydihydrochalcones of the formula (II), wherein R¹, R², R³ and R⁴ in each case have the meaning given above,
a mixture comprising or consisting of salts of two or more different 4-hydroxydihydrochalcones of the formula (11), wherein R¹, R², R³ and R⁴ in each case have the meaning given above
or
a mixture comprising or consisting of a 4-hydroxydihydrochalcone of the formula (II) or two or more different 4-hydroxydihydrochalcones of the formula (II), wherein R¹, R², R³ and R⁴ in each case have the meaning given above, and
a salt of a 4-hydroxydihydrochalcone of the formula (II) or two or more salts of different 4-hydroxydihydrochalcones of the formula (II), wherein R¹, R², R³ and R⁴ in each case have the meaning given above.

If substance (ii) is present in an aroma composition according to the invention, substance (iii) may be absent, and vice versa.

In an aroma composition according to the invention, in particular in one of the embodiments identified below as being preferred, the use of hesperetin is preferable to the use of salts of hesperetin.

An aroma composition according to the invention is advantageously used in particular to enhance the sweet flavor (in particular the initial sweetness) of a sweet-tasting substance or the sweet odor impression (in particular the initial sweetness) of an aroma substance which gives rise to a sweet odor impression.

The use of an aroma composition according to the invention to enhance the initial sweetness of a sweet-tasting substance or an aroma substance which gives rise to a sweet odor impression is most particularly advantageous.

An aroma composition according to the invention is preferred wherein:

(i) consists of one or more alkamides selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-piperide (achilleamide), 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,4E,8Z,10E,12E-tetradecapentaenoic acid-N-(2-methylpropyl)amide (γ-sanshool) and 2E,4E,8Z,11Z-tetradecatetraenoic acid-N-(2-hydroxy-2-methylpropyl)amide (bungeanool);

and/or

(ii) is hesperetin of the formula (I)

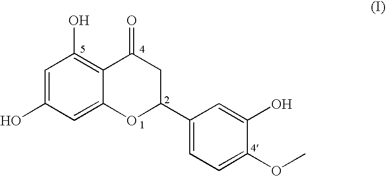

wherein the hesperetin of the formula (I) is present as the (2S)-enantiomer, (2R)-enantiomer or any desired mixture of the two enantiomers,

and/or

(iii) is a 4-hydroxydihydrochalcone of the formula (II), wherein in each case in the formula (II)

R¹ denotes OH

R² and R³, mutually independently, denote H or OH,

and

R⁴ denotes H or methoxy (OCH₃).

An aroma composition according to the invention is particularly preferred wherein:

(i) consists of one or more alkamides selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,6Z,8E-decatrienoic acid-N-isobutylamide (spilanthol), 2E,6Z,8E,10E-dodecatetraenoic acid-N-(2-methylpropyl)amide (α-sanshool), 2E,4E,8Z,11Z-tetradecatetraenoic acid-N-(2-hydroxy-2-methylpropyl)amide (bungeanool);

and/or

(ii) is hesperetin of the formula (I)

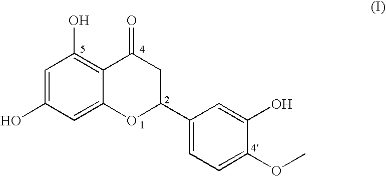

wherein the hesperetin of the formula (I) is present as the (2S)-enantiomer or any desired mixture of the two enantiomers,

and/or

(iii) consists of
a 4-hydroxydihydrochalcone of the formula (II) selected from the group consisting of:
3-(4-hydroxyphenyl)-1-(2-hydroxyphenyl)propan-1-one (2′,4-dihydroxydihydrochalcone; compound 1),
3-(4-hydroxyphenyl)-1-(2,4-dihydroxyphenyl)propan-1-one (2′,4,4′-trihydroxydihydrochalcone; davidigenin; compound 2),
3-(4-hydroxyphenyl)-1-(2,6-dihydroxyphenyl)propan-1-one (2′,4,6′-trihydroxydihydrochalcone; compound 3),
3-(4-hydroxyphenyl)-1-(2,4,6-trihydroxyphenyl)propan-1-one (2′,4,4′,6′-tetrahydroxydihydrochalcone; phloretin; compound 4),
3-(4-hydroxy-3-methoxyphenyl)-1-(2-hydroxyphenyl)propan-1-one (2′,4-dihydroxy-3-methoxydihydrochalcone; compound 5),
3-(4-hydroxy-3-methoxyphenyl)-1-(2,4-dihydroxyphenyl)propan-1-one (2′,4,4′-trihydroxy-3-methoxydihydrochalcone; compound 6),
3-(4-hydroxy-3-methoxyphenyl)-1-(2,6-dihydroxyphenyl)propan-1-one (2′,4,6′-trihydroxy-3-methoxydihydrochalcone; compound 7),
3-(4-hydroxy-3-methoxyphenyl)-1-(2,4,6-trihydroxyphenyl)propan-1-one (2′,4,4′,6′-tetrahydroxy-3-methoxydihydrochalcone; compound 8) and
3-(3,4-dihydroxyphenyl)-1-(2,4,6-trihydroxyphenyl)propan-1-one (2′,3,4,4′,6′-tetrahydroxydihydrochalcone; compound 9),
   a salt of such a 4-hydroxydihydrochalcone of the formula (II),
   a mixture comprising or consisting of two or more 4-hydroxydihydrochalcones of the formula (II) selected from said group,
   a mixture comprising or consisting of salts of two or more 4-hydroxydihydrochalcones of the formula (II) selected from said group
   or
   a mixture comprising or consisting of

one of the 4-hydroxydihydrochalcones of the formula (II) selected from said group or two or more 4-hydroxydihydrochalcones of the formula (II) selected from said group and

a salt of a 4-hydroxydihydrochalcone of the formula (II) selected from said group or two or more salts of different 4-hydroxydihydrochalcones of the formula (II) selected from said group.

An aroma composition according to the invention is most particularly preferred wherein:

(i) consists of one or more alkamides selected from the group consisting of:

2E,4E-decadienoic acid-N-isobutylamide (pellitorine), 2E,6Z,8E-decatrienoic acid-N-isobutylamide (spilanthol), 2E,6Z,8E,10E-dodecatetraenoic acid-N-(2-methylpropyl)amide (α-sanshool) and 2E,4E,8Z,11Z-tetradecatetraenoic acid-N-(2-hydroxy-2-methylpropyl)amide (bungeanool);

and/or

(ii) is hesperetin of the formula (I)

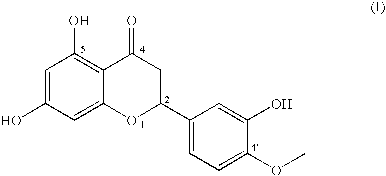

wherein the hesperetin of the formula (I) is present as the (2S)-enantiomer or any desired mixture of the two enantiomers,

and/or

(iii) consists of or comprises phloretin (compound 4).

In salts of the hesperetin of the above formula (I) or of the 4-hydroxydihydrochalcone of the above formula (II) for use according to the invention, one, several or all deprotonable groups of the hesperetin or of the 4-hydroxydihydrochalcone are deprotonated. A corresponding quantity of countercations is then present, said countercations being preferably selected from the group consisting of: unipositive cations from the first main and subgroup, ammonium ions, trialkyl ammonium ions, dipositive cations from the second main and subgroup and tripositive cations from the third main and subgroup, and mixtures thereof.

Particularly preferred cations are Na⁺, K⁺, NH₄⁺, Ca²⁺, Mg²⁺, Al³⁺ and Zn²⁺.

For the sake of clarification, the preferred compounds of the formula (II) are listed again in the illustration below (the general numbering scheme for dihydrochalcones is shown for compound 1 by way of example):

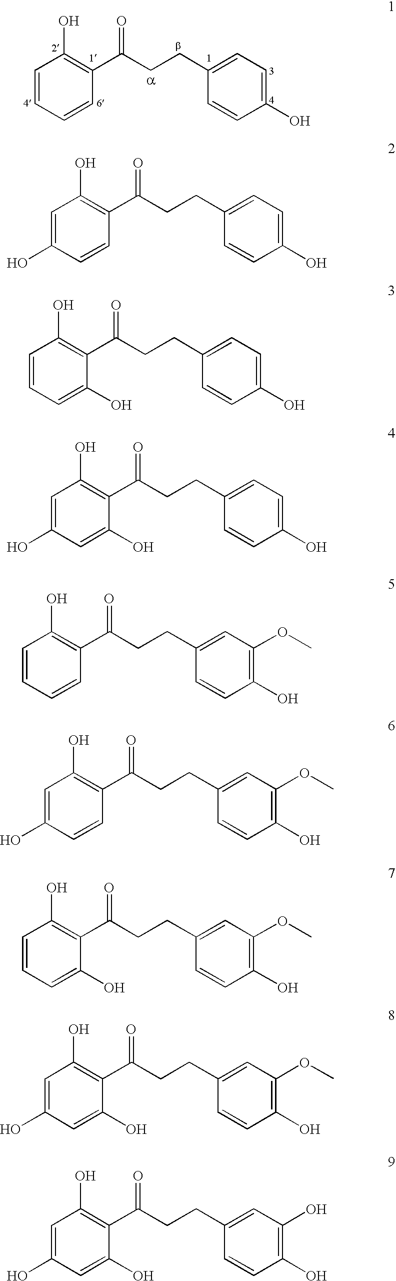

An aroma composition according to the invention is preferred wherein
the weight ratio of the total quantity of substances (i) (alkamides) to the total quantity of substances (ii) (hesperetin) and (iii) (4-hydroxydihydrochalcones) is in a range from 1:1,000,000 to 1:1, preferably in a range from 1:10,000 to 1 :10, particularly preferably in a range from 1:2000 to 1:50
and/or
the weight ratio of the total quantities of substances (ii) to substances (iii) is in the range from 1:10 to 10:1, particularly preferably in the range from 5:1 to 1:5 and especially preferably in the range from 7:3 to 3:7.

The aforementioned aroma compositions according to the invention or for use according to the invention for enhancing the sweet flavor of a sweet-tasting substance or the sweet odor impression of an aroma substance which gives rise to a sweet odor impression are preferably used in a preparation serving for nutrition, for oral care or for pleasure, in particular to enhance the initial sweetness in a preparation serving for nutrition, for oral care or for pleasure.

The invention is based on the surprising finding that not only even in very low concentrations (below 0.025 wt. %, see in this regard the concentration ranges specified further below), the aroma compositions according to the invention or for use according to the invention increase superproportionally (i.e. synergistically) the sweet flavor impression of sweet-tasting substances (as specified further below), but in particular of sugars such as sucrose, lactose, glucose, D-tagatose and fructose and sugar alcohols such as for example glycerol, erythritol, threitol, arabitol, ribitol, xylitol, sorbitol, mannitol, dulcitol and lactitol, thus making it possible to reduce the sugar content in corresponding foodstuffs and products consumed for pleasure without at the same time reducing the sweet flavor impression, but also positively influence the sweetness progression over time of the reduced-sugar application, increasing the so-called initial sweetness in particular. In low concentrations (see in this regard the preferred usage concentrations further below) the aroma compositions according to the invention or for use according to the invention exhibit only a very weak intrinsic flavor.

JP 2006-296356 describes the use of the alkamide spilanthol to enhance the general aroma impression of foodstuffs, the slight influence on the flavor value being cited as an advantage. No enhancement of the sweetness due to the presence of the alkamide, in particular no enhancement of the initial sweetness, is described. A combination of the alkamide with hesperetin or with a 4-hydroxydihydrochalcone is likewise not disclosed. It is particularly advantageous for the aroma compositions according to the invention additionally to exhibit a reducing or masking effect against unpleasant flavor notes or aftertaste notes and/or secondary flavor notes described as bitter, astringent, chalky or dry.

In addition to the specific aroma compositions already described and the use thereof, in accordance with a further aspect the invention also relates to corresponding preparations in which said aroma compositions are used in the manner according to the invention.

A preparation according to the invention is preferably selected here from the group consisting of preparations serving for nutrition, for oral care or for pleasure, semifinished products, odoriferous, aroma or flavor substance compositions or seasoning mixtures. A preparation according to the invention comprises the following components:

(a) an aroma composition according to the invention consisting of saliva-stimulating alkamides having a tingling, pungent and/or hot flavor, hesperetin and/or 4-hydroxydihydrochalcones as described further above and additionally

(b) one or more sweet-tasting substances

and/or

(c) one or more aroma substances which give rise to a sweet odor impression,

wherein the total quantity of component (a) in the preparation is sufficient to organoleptically enhance, preferably superproportionally (i.e. over and above an effect caused by intrinsic sweetness), the sweet flavor impression (in particular the initial sweetness) of the sweet-tasting substance(s) (b) or the sweet odor impression (in particular the initial sweetness) of the aroma substance(s) (c) which give rise to a sweet odor impression.

With regard to the preferred meaning of the groups R¹, R², R³ and R⁴ in formula (II), the meaning specified with regard to the use according to the invention applies correspondingly here.

In the preparations according to the invention too, the presence of an aroma composition consisting of (i) one or more saliva-stimulating alkamides having a tingling, pungent and/or hot flavor and selected from the group comprising 2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,6Z,8E-decatrienoic acid-N-isobutylamide (spilanthol), 2E,6Z,8E,10E-dodecatetraenoic acid-N-(2-methylpropyl)amide (α-sanshool) and 2E,4E,8Z,11Z-tetradecatetraenoic acid-N-(2-hydroxy-2-methylpropyl)amide (bungeanool) and (ii) hesperetin ([S]— or [R]-enantiomer or mixtures thereof) and/or (iii) a 4-hydroxydihydrochalcone of the formula (II) selected from the group consisting of the aforementioned compounds 1 to 9, a corresponding salt or a corresponding mixture (as specified in the details above) is preferred.

It may be mentioned at this point that all details of preferred embodiments of an aroma composition according to the invention, a use according to the invention, a preparation according to the invention or a method according to the invention apply correspondingly in each case to the other aspects of the invention.

A preferred preparation according to the invention comprises as component (b) one or more sugars, the total quantity of component (a) in the preparation being sufficient to impart, in comparison to a preparation or a semifinished product which, having an otherwise identical composition, contains no component (a) but at least 1.05 times the quantity (preferably at least 1.2 times, by preference 1.4 times the quantity) of sugar, the same or an enhanced sweetness impression, in particular in the initial sweetness. The sugars are preferably selected here from the group consisting of: sucrose, lactose, glucose, fructose and mixtures thereof.

Regarding the occurrence and the production of the substances cited under (i), (ii) and (iii), reference may be made in respect of (i) to WO 2004/000787, WO 2004/043960 and in respect of (ii) and (iii) to WO 2007/014879. The preferred mixtures of pellitorines described in WO 2004/043960 are likewise preferred for the purposes of the present invention.

For in particular the saliva-stimulating alkamides having a tingling, pungent and/or hot flavor from group (i), natural sources are also known and able to be used. Preferred sources are plant extracts such as for example alkamide-containing pepper extract (Piper ssp., in particular P. nigrum, P. hispidum, P. tuberculatum, P. longum, P. arboreum, P. futokadsura, P. guineense, P. sarmentosum or Piper nigrum, Var. muntok, P. aff pedicellatum), extracts of toothache grass (Ctenium aromaticum), extracts of tarragon (Artemisia dracunculus), pellitory root extracts (Anacyclus ssp., in particular Anacyclus pyrethrum L.), coneflower extracts (Echinaceae ssp., for example E. angustifolia), extracts of Szechuan pepper (Zanthoxylum ssp., in particular Zanthoxylum piperitum, Z. clava-herculis, Z. bungeanum, Z. zanthoxyloides), spilanthes extract (Spilanthes ssp., in particular Spilanthes acmella), extracts of Acmella ssp. (for example A. ciliata), extracts of Achillea ssp. (for example Achillea wilsoniana), extracts of fagara species (Fagara zanthoxyloides), extracts of Heliopsis ssp. (for example H. longipes), extracts of Cissampelos glaberrima, extracts of Dinosperma erythrococca, extracts of the bark of Esenbeckia alata and extracts of Stauranthus perforatus.

The plant extracts can be obtained from the corresponding fresh or dried plants or plant parts, but in particular from white, green or black peppercorns (P. nigrum), long pepper (P. longum), coneflower roots, pellitory root, Szechuan pepper, plant parts of the other Zanthoxylum species, plant parts of the Spilanthes or Acmella species, plant parts of the Fagara or Heliopsis species. The dried plant parts (for example fresh or dried roots, fruits, seeds, bark, wood, stems, leaves or flower [parts]), preferably in comminuted form, are conventionally extracted with a solvent suitable for foodstuffs and for products consumed for pleasure at temperatures of 0° C. up to the boiling point of the individual solvent or solvent blend, then filtered, and the filtrate is wholly or partially concentrated to small volume, preferably by distillation, freeze drying or spray drying. The raw extract thus obtained can then be further processed, for example steam treated, mostly under pressures of 0.01 mbar to normal pressure, and/or taken up in a solvent suitable for foodstuffs and for products consumed for pleasure.

A solvent suitable for extraction for foodstuffs and of products consumed for pleasure is preferably selected from the group consisting of: water, ethanol, methanol, propylene glycol, glycerol, acetone, dichloromethane, ethyl acetate, diethyl ether, hexane, heptane, triacetine, vegetable oils or fats, supercritical carbon dioxide and mixtures thereof.

A preferred preparation according to the invention (as described above, in particular in a preferred embodiment) comprises

(b) one or more further sweet-tasting substances, wherein the further sweet-tasting substance(s) are selected from the group consisting of:
one or more carbohydrates (sugars) selected from the group consisting of sucrose, trehalose, lactose, maltose, melizitose, melibiose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin and plant preparations containing one or more of the cited carbohydrates (preferably in a proportion of at least 5 wt. %, preferably at least 15 wt. %), wherein these carbohydrates can also be present as a natural or artificially produced mixture (for example as honey, invert sugar syrup, highly concentrated fructose syrups made from corn starch [high fructose corn syrup])
one or more sugar alcohols selected from the group consisting of glycerol, erythritol, threitol, arabitol, ribitol, xylitol, sorbitol, mannitol, maltitol, isomaltitol, dulcitol and lactitol,
one or more proteins and/or amino acids from the group consisting of miraculin, monellin, thaumatin, curculin, brazzein, glycine, D-leucine, D-threonine, D-asparagine, D-phenylalanine, D-tryptophan, L-proline or extracts or fractions containing these amino acids and/or proteins obtained from natural sources
one or more sweeteners from the group consisting of MAGAP, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatin and phyllodulcin, wherein in the case of naturally occurring sweeteners extracts or concentrated fractions of these extracts can also be used, for example thaumatococcus extracts (katemfe plant), stevia extracts, citrus extracts, buddha tea extracts

and mixtures thereof

and/or

(c) one or more (optionally further) aroma substances which give rise to a sweet odor impression, wherein the further aroma substance(s) which give rise to a sweet odor impression are selected from the group consisting of:

vanillin, ethyl vanillin, ethyl vanillin isobutyrate (=3-ethoxy-4-isobutyryloxybenzaldehyde), Furaneol® (2,5-dimethyl-4-hydroxy-3(2H)-furanone) and derivatives (for example homofuraneol, 2-ethyl-4-hydroxy-5-methyl-3(2H)-furanone), homofuronol (2-ethyl-5-methyl-4-hydroxy-3(2H )-furanone and 5-ethyl-2-methyl-4-hydroxy-3(2H)-furanone), maltol and derivatives (for example ethyl maltol), coumarin and derivatives, gamma-lactones (for example gamma-undecalactone, gamma-nonalactone), delta-lactones (for example 4-methyl delta-lactone, massoilactone, delta-decalactone, tuberolactone), methylsorbate, divanillin, 4-hydroxy-2(or 5)-ethyl-5(or 2)-methyl-3(2H)furanone, 2-hydroxy-3-methyl-2-cyclopentenones, 3-hydroxy-4,5-dimethyl-2(5H)-furanone, fruit esters and fruit lactones (for example acetic acid-n-butyl ester, acetic acid isoamyl ester, propionic acid ethyl ester, butyric acid ethyl ester, butyric acid-n-butyl ester, butyric acid isoamyl ester, 3-methyl butyric acid ethyl ester, n-hexanoic acid ethyl ester, n-hexanoic acid allyl ester, n-hexanoic acid-n-butyl ester, n-octanoic acid ethyl ester, ethyl-3-methyl-3-phenyl glycidate, ethyl-2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenyl acetaldehyde.

Preference is given to the use of sweet-tasting substances selected from the group consisting of

(a) sucrose, lactose, D-glucose, D-tagatose and D-fructose, wherein these carbohydrates can also be present as a natural or artificially produced mixture (for example as honey, invert sugar syrup, highly concentrated fructose syrups from corn starch [high fructose corn syrup])

(b) erythritol, threitol, arabitol, ribitol, xylitol, sorbitol, mannitol, maltitol, isomaltitol, dulcitol and lactitol,

(c) thaumatin, glycine, D-phenylalanine, D-tryptophan,

(d) sweeteners from the group of sodium cyclamate, acesulfame K, neohesperidin dihydrochalcone, saccharin sodium salt, aspartame, superaspartame, neotame, alitame, sucralose, stevioside,

wherein the quantity of added aroma composition according to the invention in the preparation is sufficient to organoleptically enhance the sweet flavor impression of the sweet-tasting substance(s), in particular the initial sweetness; the total quantity of the aroma composition according to the invention here is preferably in the range from 0.1 to 150 ppm, preferably in the range from 1 to 50 ppm, particularly preferably in the range from 10 to 50 ppm, relative to the total weight of the preparation.

In particular, a synergistic increase in the sweet flavor impression (as mentioned above) can be obtained with this combination.

Particular preference is given to a preparation as defined above and serving for nutrition, for oral care or for pleasure (in particular in one of the embodiments identified as being preferred) comprising a total quantity in the range from 0.1 to 150 ppm, preferably in the range from 1 to 50 ppm, particularly preferably in the range from 10 to 50 ppm, of an aroma composition as defined above (in particular in one of the embodiments identified as being preferred), relative to the total weight of the preparation.

Preferred sweet-tasting substances were specified above. As a general rule, however, sweet-tasting substances (including natural sources of said substances) can be, for example: sweet-tasting carbohydrates or sugars (for example sucrose (synonym for saccharose), trehalose, lactose, maltose, melizitose, melibiose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin) or plant preparations containing predominantly said carbohydrates (for example from sugar beet (Beta vulgaris ssp., sugar fractions, sugar syrup, molasses), from sugar cane (Saccharum officinarum ssp., for example molasses, sugar syrups), from sugar maple (Acer ssp.), from agave (agave nectar), artificial/enzymatic hydrolysates of starch or sucrose (for example invert sugar syrup, highly concentrated fructose syrups from corn starch), fruit concentrates (for example from pears, pear concentrate), sugar alcohols (for example glycerol, erythritol, threitol, arabitol, ribitol, xylitol, sorbitol, mannitol, maltitol, isomaltitol, dulcitol, lactitol), proteins (for example miraculin, monellin, thaumatin, curculin, brazzein), sweeteners (MAGAP, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatin, phyllodulcin), certain sweet-tasting amino acids (glycine, D-leucine, D-threonine, D-asparagine, D-phenylalanine, D-tryptophan, L-proline), other sweet-tasting low-molecular-weight substances (for example hernandulcin, isocoumarins such as phyllodulcin or hydrangenol, dihydrochalcone glycosides such as neohesperidin dihydrochalcone, glycyrrhizin, glycyrrhetinic acid ammonium salt or other glycyrrhetinic acid derivatives), extracts of liquorice (Glycyrrhizza glabra ssp.), extracts of Lippia dulcis, extracts or single substances of Momordica ssp. (in particular Momordica grosvenori [luo han guo] and the mogrosides obtained therefrom, extracts of Thaumatococcus danielli (katemfe plant), of Hydrangea dulcis or of Stevia ssp. (for example Stevia rebaudiana).

The aforementioned preferred aroma substances are aroma substances which give rise to a sweet odor impression, i.e. aroma substances which while not tasting sweet in the narrower definition suggest a sweet flavor in the wider definition (including odor perception in particular).

Preference is given to a preparation according to the invention, in particular a preparation serving for nutrition, for oral care or for pleasure, wherein the total quantity
of substances (i) (saliva-stimulating alkamides having a tingling, pungent and/or hot flavor) is in the range from 0.005 to 5 ppm, preferably 0.02 to 2 ppm, particularly preferably 0.05 to 0.5 ppm,
of substances (ii) (hesperetin as defined further above) is in the range from 0.5 to 500 ppm, preferably 10 to 200 ppm, particularly preferably 20 to 100 ppm,
and/or
of substances (iii) (4-hydroxydihydrochalcones as defined further above) is in the range from 0.5 to 500 ppm, preferably 5 to 100 ppm, particularly preferably 10 to 40 ppm,
   wherein the total quantity of all components (i), (ii) and (iii) is in the range from 0.5 to 500 ppm, preferably in the range from 5 to 200 ppm, particularly preferably in the range from 10 to 100 ppm. The specified concentrations relate here to the finished preparation, for example the ready-to-consume preparation, containing the aroma composition according to the invention.

Aroma compositions according to the invention are preferably used in particular to enhance the initial sweetness of a sweet-tasting substance or the sweet odor impression of an aroma substance which gives rise to a sweet odor impression, in a preparation serving for nutrition, for oral care or for pleasure.

Through the use of the aroma compositions according to the invention it is possible in particular to reduce the proportion of sweet-tasting substances, but in particular of sugars such as sucrose, lactose, fructose and/or glucose or mixtures thereof, by 5 to 60% (relative to the sweet-tasting substance(s)) as compared with a preparation not containing the aroma composition according to the invention, without the sweet flavor impression being reduced as a consequence.

Preference is therefore given to (reduced-sugar) preparations according to the invention which comprise as sweet-tasting substance or as sweet-tasting substances of component (b) one or more sugars, the quantity of added aroma composition according to the invention being sufficient to impart, in comparison to a preparation which, having an otherwise identical composition, contains no aroma composition according to the invention but at least 1.05 times, preferably at least 1.2 times, particularly preferably 1.4 times the quantity of sugar, the same or an enhanced sweetness impression, in particular in the initial sweetness. The sugars are preferably selected here from the group consisting of: sucrose, lactose, glucose, fructose and mixtures thereof.

Preferred preparations according to the invention, which can be sugar-free, reduced-sugar or sugar-containing and which are preferably serving for nutrition, for oral care or for pleasure, are selected from the group consisting of:
(A) confectionery, for example white, milk or dark chocolates, filled chocolates (filled for example with aromatized fondant mass of the After Eight type), chocolate bars, other bar products, chewing candies, fruit gums, hard-boiled and soft candies, chewing gum, sugar pearls, lollipops), capsules (preferably seamless capsules for direct consumption, preferably with a shell based on gelatin and/or alginate), chewing gum (for example in the form of strips, tablets, pellets, dragees, balls, hollow balls),
(B) alcoholic or non-alcoholic beverages or instant beverages, in particular coffee, tea, wine, wine-based beverages, beer, beer-based beverages, liqueurs, spirits, brandies, fruit-based carbonated beverages, isotonic beverages, soft drinks, nectars, fruit and vegetable juices, fruit or vegetable juice preparations, instant cocoa beverages, instant tea beverages, instant coffee beverages,
(C) cereal products and/or nut products, in particular breakfast cereals, cornflakes, oat flakes, muesli, muesli bars, trail mix, sweet popcorn, nut bars, fruit and nut bars, precooked ready rice products,
(D) dairy products, in particular milk beverages, milk ice cream, diet ice cream, yogurt, custard, kefir, curd cheese, soft cheese, hard cheese, dried milk powder, whey, butter, buttermilk, products containing partially or entirely hydrolyzed milk protein,
(E) products made from soya protein or other soya bean fractions (for example soya milk and products produced therefrom, preparations containing soya lecithin, fermented products such as tofu or tempeh or products produced therefrom),
(F) fruit and/or vegetable preparations, in particular jams, diabetic jams, fruit ice cream, fruit sauces, fruit fillings, ketchup, sauces, dried vegetables, deep-frozen vegetables, precooked vegetables, pickled vegetables, preserved vegetables,
(G) fat- and oil-based products or emulsions thereof, in particular mayonnaise, remoulade, dressings, seasoning preparations,
(H) oral care products (oral hygiene products), in particular in the form of toothpaste, tooth cream, tooth gel, tooth powder, dental cleaning liquid, dental cleaning foam, mouthwash, mouthwash concentrate, tooth cream and mouthwash as a 2-in-1 product, hard candies, mouth spray, dental floss, chewing gums or dental care chewing gums.

Of particular significance is thus a preparation according to the invention, comprising at least one sweet-tasting substance, preferably a sugar such as sucrose, lactose, glucose and/or fructose, wherein the quantity of the sweet-tasting substance is not sufficient to impart a satisfactory sweet taste in a comparative preparation not containing an aroma composition according to the invention but having an otherwise identical composition, wherein the quantity of the aroma composition according to the invention that is present in the preparation is sufficient to organoleptically enhance the sweet flavor impression of the sweet-tasting substance, preferably to such an extent that a satisfactory initial sweetness is imparted.

Preferred preparations according to the invention are preparations serving for nutrition, for oral care or for pleasure, with regard to whose compositions the aforementioned details apply.

The preparations according to the invention serving for nutrition, for oral care or for pleasure are generally products which are intended to be introduced into the human oral cavity, to remain there for a certain time and then either to be consumed (for example ready-to-consume foodstuffs) or removed from the oral cavity again (for example chewing gums or toothpaste). It goes without saying that the use of the aroma compositions according to the invention is intended for any type of such products. These products include all substances or products which are intended to be introduced by a person into the oral cavity in the processed, semi-processed or unprocessed state. This also includes substances which are added to foodstuffs during their production, processing or finishing and which are intended to be introduced into the human oral cavity.

It goes without saying that the aroma compositions according to the invention can be used in particular in foodstuffs. Within the meaning of the present text, a “foodstuff” is understood to refer in particular to substances which are intended to be swallowed by a person in an unchanged, prepared or processed state and then digested; the term “foodstuff” thus also covers shells, coatings or other encapsulations which are intended to be swallowed at the same time or for which swallowing is anticipated. Certain products which are conventionally removed again from the oral cavity (for example chewing gums) are also understood to be foodstuffs within the meaning of the present text, since the possibility of at least a part thereof being swallowed cannot be excluded.

In particular, the aroma compositions according to the invention are used in ready-to-consume foodstuffs. A ready-to-consume foodstuff is understood here to be a foodstuff which is already fully constituted in terms of the substances that are critical to its flavor. The term “ready-to-consume foodstuff” also includes corresponding beverages and solid or semi-solid ready-to-consume foodstuffs. Examples include deep-frozen products which have to be thawed and heated to eating temperature before being consumed. Products such as yogurt or ice cream as well as chewing gums or hard-boiled candies are also classed as ready-to-consume foods.

The aroma compositions according to the invention can also be used in semifinished foodstuff products. The term “semifinished foodstuff product” relates here to foodstuffs which are intended to be consumed only in the further processed state, after addition of aroma or flavor substances which determine or help to determine the sensory impression.

A preparation serving for oral care (oral care product, also known as oral hygiene product or oral hygiene preparation) within the meaning of the invention is understood to be a formulation for cleansing and care of the oral cavity and pharynx and for freshening the breath. This expressly includes care of the teeth and gums. Forms for administration of common oral hygiene formulations are creams, gels, pastes, foams, emulsions, suspensions, aerosols, sprays and also capsules, granules, pastilles, tablets, candies or chewing gums, this list not being intended to be limiting for the purposes of this invention.

Further conventional active ingredients, basic materials, auxiliary substances and additives for preparations according to the invention serving for nutrition, for oral care or for pleasure may be present in quantities of 5 to 99.999999 wt. %, preferably of 10 to 80 wt. %, relative to the total weight of the preparation. The preparations may furthermore comprise water in a quantity of up to 99.999999 wt. %, preferably of 5 to 80 wt. %, relative to the total weight of the preparation.

The present invention relates in particular to a preparation serving for nutrition, for oral care or for pleasure comprising

a component (a) consisting of the aroma composition according to the invention,

a component (b) (i.e. one or more sweet-tasting substances) comprising or consisting of one or more sugars,

and optionally

component (c) (i.e. one or more aroma substances which give rise to a sweet odor impression)

wherein the total quantity of component (a) in the preparation

is sufficient to impart, in comparison to a preparation which, having an otherwise identical composition, contains none of the aroma compositions according to the invention but at least 1.05 times the quantity of sugar, the same or an enhanced sweetness impression

and/or

is in the range from 0.5 to 500 ppm.

Further preferred preparations according to the invention are semifinished products, odoriferous substance compositions, other aroma or flavor substance compositions or seasoning mixtures.

The term “semifinished products” includes in particular here semifinished foodstuff products as defined above. Semifinished products according to the invention are preferably in spray-dried form. Preparations according to the invention may also be nutritional supplements in the form of capsules, tablets (uncoated and coated tablets, for example coatings resistant to gastric juices), sugar-coated tablets, granules, pellets, mixtures of solids, dispersions in liquid phases, as emulsions, as powders, as solutions, as pastes or as other swallowable or chewable preparations.

Preparations according to the invention, which are present as semifinished products, can be used in particular to enhance the sweet flavor impression of finished product preparations serving for nutrition, for oral care or for pleasure which are produced using the semifinished product preparation.

Spray-dried solid preparations according to the invention as semifinished products are particularly well suited to producing preparations according to the invention which can serve in particular for nutrition, for oral care or for pleasure. In the spray-dried semifinished products the solubility of the aroma compositions according to the invention is in particular substantially improved by the carriers and/or auxiliary substances, in particular by maltodextrin, starch, natural or artificial polysaccharides and/or vegetable gums such as modified starches or gum arabic. The spray-dried solid semifinished products according to the invention preferably contain 50 to 95 wt. % carriers, in particular maltodextrin and/or starch, 5 to 40 wt. % auxiliary substances, preferably natural or artificial polysaccharides and/or vegetable gums such as modified starches or gum arabic, and 1 to 45 wt. % aroma compositions according to the invention, relative to the total quantity of the spray-dried solid preparation.

Preparations according to the invention selected from the group consisting of semifinished products, odoriferous, aroma or flavor substance compositions or seasoning mixtures preferably comprise a total quantity in the range from 0.0001 wt. % to 95 wt. %, preferably 0.001 wt. % to 80 wt. %, particularly preferably 0.001 wt. % to 50 wt. %, of aroma compositions according to the invention, relative to the total weight of the preparation.

The preparations according to the invention may also contain further aroma compositions in order to round off and refine the flavor and/or odor of the preparation. Suitable further aroma compositions contain for example synthetic, natural or nature-identical aroma, odoriferous and flavor substances and suitable auxiliary substances and carriers. Preparations according to the invention containing one or more aroma substances which give rise to a “sweet” odor impression (see above in this regard) are particularly preferred. Preferred further synthetic, natural or nature-identical aroma, odoriferous and flavor substances are further saliva-stimulating substances having a tingling, pungent and/or hot flavor such as for example culinary acids (for example citric acid, malic acid, tartaric acid), capsaicin, dihydrocapsaicin, gingerols, paradols, shogaols, piperine, carboxylic acid-N-vanillylamides, in particular nonanoic acid-N-vanillylamide, 2-alkenoic acid amides, in particular 2-nonenoic acid-N-isobutylamide, 2-nonenoic acid-N-4-hydroxy-3-methoxyphenylamide, alkyl ethers of 4-hydroxy-3-methoxybenzyl alcohol, in particular 4-hydroxy-3-methoxybenzyl-n-butyl ether, alkyl ethers of 4-acyloxy-3-methoxybenzyl alcohol, in particular 4-acetyloxy-3-methoxybenzyl-n-butyl ether and 4-acetyloxy-3-methoxybenzyl-n-hexyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylene dioxybenzyl alcohol, (4-hydroxy-3-methoxyphenyl) acetic acid amides, in particular (4-hydroxy-3-methoxyphenyl)acetic acid-N-n-octylamide, ferulic acid phenethyl amides, in particular N-feruloyl-3-methoxytyramine or rubemamine, nicotinaldehyde, methyl nicotinate, propyl nicotinate, 2-butoxyethyl nicotinate, benzyl nicotinate, 1-acetoxychavicol, polygodial, isodrimeninol and pilocarpin.

Semifinished products according to the invention regularly comprise further flavor and/or aroma substances, in particular aroma substances which give rise to a sweet odor impression (see further above), and suitable solvents (for example ethanol, glycerol, 1,2-propylene glycol, alkyl esters of lactic acid, ethyl esters of organic fruit acids such as diethyl malonate, diethyl tartrate, diethyl malate, triethyl citrate, diethyl succinate, diethyl fumarate, diethyl maleate) and further auxiliary substances (for example dyes, pigments, antioxidants, preservatives, emulsifiers, viscosity-influencing substances).

Spray-dried solid semifinished products according to the invention preferably comprise 1 to 50 wt. % aroma composition according to the invention, relative to the total weight of the preparation, 0 to 10 wt. %, preferably 1 to 10 wt. % other aromas, 50 to 99 wt. % carriers and 0 to 50 wt. %, preferably 1 to 50 wt. % other auxiliary substances and/or stabilizers, relative in each case to the total weight of the preparation.

Advantageous carriers in the spray-dried solid preparations according to the invention are carbohydrates and/or carbohydrate polymers (polysaccharides). Preferred examples of carriers in the aroma particles for use according to the invention are hydrocolloids such as starches, degraded starches, chemically or physically modified starches, modified celluloses, gum arabic, gum ghatti, gum tragacanth, karaya, carrageenan, guar gum, locust bean gum, alginates, pectin, inulin or xanthan gum, dextrins and maltodextrins.

The degree of decomposition of the starch is measured by the “dextrose equivalent” value (DE), which can assume the limiting values 0 for the long-chain glucose polymer starch and 100 for pure glucose.

Particularly preferred carriers for the spray-dried solid preparations according to the invention are maltodextrins, maltodextrins having DE values in the range from 10 to 30 being advantageous here.

It has already been mentioned that spray-dried solid semifinished products are particularly well suited to producing preparations according to the invention which are to serve for nutrition, for oral care or for pleasure.

A preferred preparation according to the invention comprises as additional component (d) one or more esters selected from the group consisting of lactic acid-C₁-C₆-esters, tartaric acid di-C₁-C₄-esters, succinic acid di-C₁-C₄-esters, malonic acid di-C₁-C₄-esters, malic acid di-C₁-C₄-esters, citric acid di-C₁-C₄-esters and citric acid tri-C₁-C₄-esters,

and/or

one or more solvents selected from the group consisting of 1,2-propylene glycol, dimethyl sulfoxide, ethanol and ethanol/water blends.

In addition to the additional component (d), one or more further aroma substances are preferably present, in particular aroma substances which give rise to a sweet odor impression and which are preferably selected from the aforementioned group of such aroma substances.

Particularly preferred for use in the aroma compositions according to the invention are esters selected from the group consisting of ethyl lactate, n-propyl lactate, n-butyl lactate, diethyl tartrate, dimethyl succinate, diethyl succinate, dimethyl malonate, diethyl malonate, dimethyl malate, diethyl malate and triethyl citrate and the solvent 1,2-propylene glycol.

The presence of the aforementioned esters or solvents in aroma compositions according to the invention brings about a very good solubility and prevents any significant tendency to recrystallization of the aroma compositions according to the invention. Such aroma compositions according to the invention are therefore particularly suitable for incorporation into preparations according to the invention serving for nutrition, for oral care or for pleasure. With regard to the preferred concentrations of the aroma compositions according to the invention, reference may be made to the details given above.

Preparations according to the invention serving for nutrition, for oral care or for pleasure are preferably produced by incorporating the individual substances (i), (ii) and/or (iii) of the aroma compositions according to the invention as pure substances, as a solution (for example in ethanol, water, 1,2-propylene glycol, dimethyl sulfoxide, optionally in the presence of one of the aforementioned esters or solvents) or in the form of a mixture with a solid or liquid carrier (for example maltodextrin, starch, silica gel), further aromas or aroma substances and optionally further auxiliaries and/or stabilizers (for example natural or artificial polysaccharides and/or vegetable gums such as modified starches or gum arabic), i.e. in the form of a semifinished product, into a base preparation serving for nutrition, for oral care or for pleasure. Advantageously, a preparation assuming solution and/or suspension or emulsion form may first be converted by spray drying into a solid preparation according to the invention (semifinished product) before said solid preparation is then used to produce a preparation according to the invention serving for nutrition, for oral care or for pleasure. Regarding the special suitability of spray-dried semifinished products for producing preparations serving for nutrition, for oral care or for pleasure, reference may be made to the details given above.

According to a further preferred embodiment, preparations according to the invention are produced by initially incorporating the aroma compositions according to the invention and optionally other constituents of the preparation according to the invention into emulsions, into liposomes, for example starting from phosphatidyl choline, into microspheres, into nanospheres or also into capsules, granules or extrudates made from a matrix suitable for foodstuffs and products consumed for pleasure, for example from starch, starch derivatives (for example modified starch), cellulose or cellulose derivatives (for example hydroxypropylcellulose), other polysaccharides (for example dextrin, alginate, curdlan, carageenan, chitin, chitosan, pullulan), natural fats, natural waxes (for example beeswax, carnauba wax), from proteins, for example gelatin, or other natural products (for example shellac). In said embodiment, depending on the matrix, the products may be obtained by spray drying, spray granulation, melt granulation, fluidized bed methods (for example in accordance with WO 97/16078 or WO 2004/022642), fluidized bed spray granulation (for example in accordance with WO 00/36931 or U.S. Pat. No. 4,946,654), coacervation, coagulation, extrusion, melt extrusion (for example in accordance with WO 2003/092412, EP 1 123 660 or EP 1 034 705), emulsion methods, coating or other suitable encapsulation methods and optionally a suitable combination of the aforementioned methods. In a further preferred production method for a preparation according to the invention, the aroma compositions according to the invention are initially complexed with one or more suitable complexing agents, for example with cyclodextrins or cyclodextrin derivatives, preferably alpha- or beta-cyclodextrin, and in used in this complexed form.

In some cases a preparation according to the invention is preferred in which the matrix is selected such that the aroma composition according to the invention is released in delayed manner from the matrix, such that a long-lasting action is achieved. A fat, wax, polysaccharide or protein matrix is particularly preferred in this respect.

Further constituents for preparations serving for nutrition or for pleasure according to the invention which may be used are conventional basic materials, auxiliary substances and additives for foodstuffs or products consumed for pleasure, for example water, mixtures of fresh or processed, plant or animal basic or raw materials (for example raw, roasted, dried, fermented, smoked and/or boiled meat, bone, cartilage, fish, vegetables, fruit, herbs, nuts, vegetable or fruit juices or pastes or mixtures thereof), digestible or non-digestible carbohydrates (for example amylose, amylopectin, inulin, xylans, cellulose), natural or hydrogenated fats (for example tallow, lard, palm fat, coconut oil, hydrogenated vegetable fat), oils (for example sunflower oil, peanut oil, maize germ oil, olive oil, fish oil, soya oil, sesame oil), fatty acids or the salts thereof (for example potassium stearate), proteinogenic or non-proteinogenic amino acids and related compounds (for example y-aminobutyric acid, taurine), peptides (for example glutathione), native or processed proteins (for example gelatin), enzymes (for example peptidases), nucleic acids, nucleotides, flavor-correcting agents for unpleasant flavor impressions, further flavor modulators for further generally not unpleasant flavor impressions, other flavor-modulating substances (for example inositol phosphate, nucleotides such as guanosine monophosphate, adenosine monophosphate or other substances such as sodium glutamate or 2-phenoxypropionic acid), emulsifiers (for example lecithins, diacylglycerols, gum arabic), stabilizers (for example carrageenan, alginate), preservatives (for example benzoic acid, sorbic acid), antioxidants (for example tocopherol, ascorbic acid), chelating agents (for example citric acid), organic or inorganic acidulants (for example malic acid, acetic acid, citric acid, tartaric acid, phosphoric acid), bitter substances (for example quinine, caffeine, limonene, amarogentin, humolone, lupolone, catechins, tannins), mineral salts (for example sodium chloride, potassium chloride, magnesium chloride, sodium phosphates), substances preventing enzymatic browning (for example sulfite, ascorbic acid), essential oils, plant extracts, natural or synthetic dyes or coloring pigments (for example carotenoids, flavonoids, anthocyans, chlorophyll and the derivatives thereof), spices, trigeminally active substances or plant extracts containing such trigeminally active substances, cooling active ingredients such as for example menthol, menthol derivatives (for example L-menthol, L-menthyl lactate, L-menthyl glutarate, L-menthyl succinate) or cubebol, synthetic, natural or nature-identical aroma substances or odoriferous substances and odor-correcting agents.

Dental care products (as an example of an oral care product according to the invention) which contain an aroma composition according to the invention generally comprise an abrasive system (abrasive or polishing agent), such as for example silicas, calcium carbonates, calcium phosphates, aluminum oxides and/or hydroxylapatites, surface-active substances such as for example sodium lauryl sulfate, sodium lauryl sarcosinate and/or cocamidopropyl betaine, humectants such as for example glycerol and/or sorbitol, thickeners, such as for example carboxymethylcellulose, polyethylene glycols, carrageenan and/or Laponite®, sweeteners, such as for example saccharin, flavor-correcting agents for unpleasant flavor impressions, flavor-correcting agents for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), cooling active ingredients such as for example menthol, menthol derivatives (for example L-menthol, L-menthyl lactate, L-menthyl alkylcarbonates, menthone ketals, menthane carboxamides, L-menthyl glutarate, L-menthyl succinate), 2,2,2-trialkylacetamides (for example 2,2-diisopropyl propionic acid methylamide), icilin and icilin derivatives, stabilizers and active ingredients, such as for example sodium fluoride, sodium monofluorophosphate, tin difluoride, quaternary ammonium fluorides, zinc citrate, zinc sulfate, tin pyrophosphate, tin dichloride, mixtures of different pyrophosphates, triclosan, cetylpyridinium chloride, aluminum lactate, potassium citrate, potassium nitrate, potassium chloride, strontium chloride, hydrogen peroxide, aromas and/or sodium bicarbonate or odor-correcting agents.

Chewing gums (as a further example of the preparations for oral care purposes) which contain the aroma compositions according to the invention generally comprise a chewing gum base, i.e. a chewable mass which becomes plastic on chewing, sugars of various kinds, sugar substitutes, other sweet-tasting substances, sugar alcohols, flavor-correcting agents for unpleasant flavor impressions, other flavor modulators for further, generally not unpleasant flavor impressions, flavor-modulating substances (for example inositol phosphate, nucleotides such as guanosine monophosphate, adenosine monophosphate or other substances such as sodium glutamate or 2-phenoxypropionic acid), cooling active ingredients such as for example menthol, menthol derivatives (for example L-menthol, L-menthyl lactate, L-menthylalkyl carbonates, menthone ketals, menthane carboxamides, L-menthyl glutarate, L-menthyl succinate), 2,2,2-trialkylacetamides (for example 2,2-diisopropyl propionic acid methylamide), icilin and icilin derivatives, caffeine, theobromine, theophylline, humectants, thickeners, emulsifiers, aromas and stabilizers or odor-correcting agents.

A particularly preferred preparation according to the invention comprises at least one further substance for masking or reducing a bitter, metallic, chalky, acidic or astringent taste impression or for enhancing a sweet, salty or umami taste impression. The aroma compositions according to the invention are thus used in combination with at least one (further) substance suitable for masking or reducing an unpleasant (bitter, metallic, chalky, acidic, astringent) flavor impression or for enhancing a pleasant flavor impression (sweet, salty, umami). These special preparations are outstandingly suitable for achieving a particularly effective enhancement of the initial sweetness in the preparations according to the invention containing sweet-tasting substances. Particular preference is given to the combination of the aroma compositions according to the invention with flavor-correcting agents for unpleasant, in particular bitter, flavor impressions or flavor enhancers for pleasant, in particular sweet, flavor impressions.

The (further) flavor-correcting agents are selected from the following list, for example: nucleotides (for example adenosine-5′-monophosphate, cytidine-5′-monophosphate) or the pharmaceutically acceptable salts thereof, lactisoles, sodium salts (for example sodium chloride, sodium lactate, sodium citrate, sodium acetate, sodium gluconoate), further hydroxyflavanones (for example eriodictyol, homoeriodictyol or the sodium salts thereof), in particular in accordance with US 2002/0188019, hydroxybenzoic acid amides in accordance with DE 10 2004 041 496 (for example 2,4-dihydroxybenzoic acid vanillylamide, 2,4-dihydroxybenzoic acid-N-(4-hydroxy-3-methoxybenzyl)amide, 2,4,6-trihydroxybenzoic acid-N-(4-hydroxy-3-methoxybenzyl)amide, 2-hydroxy-benzoic acid-N-4-(hydroxy-3-methoxybenzyl)amide, 4-gydroxybenzoic acid-N-(4-hydroxy-3-methoxybenzyl)amide, 2,4-dihydroxybenzoic acid-N-(4-hydroxy-3-methoxybenzyl)amide monosodium salt, 2,4-dihydroxybenzoic acid-N-2-(4-hydroxy-3-methoxyphenyl)ethylamide, 2,4-dihydroxybenzoic acid-N-(4-hydroxy-3-ethoxybenzyl)amide, 2,4-dihydroxybenzoic acid-N-(3,4-dihydroxybenzyl)amide and 2-hydroxy-5-methoxy-N-[2-(4-hydroxy-3-methoxyphenyl)ethyl]amide (aduncamide), 4-hydroxybenzoic acid vanillylamide), bitterness-masking hydroxydeoxybenzoins in accordance with W02006/106023 and the documents (Symrise) based thereon (for example 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl)ethanone), amino acids (for example gamma-aminobutyric acid in accordance with WO 2005/096841 for reducing or masking an unpleasant flavor impression such as bitterness), malic acid glycosides in accordance with WO 2006/003107, salty-tasting mixtures in accordance with U.S. Provisional Application 60/728,744 and the documents (Symrise) based thereon (PCT/EP2006/067120), diacetyl trimers in accordance with PCT/EP 2005/056355 and the documents (Symrise) based thereon, divanillin, mixtures of whey proteins with lecithins and/or bitterness-masking substances such as ginger diones in accordance with WO2007/003527.

It has already repeatedly been stated that preparations according to the invention are selected here in particular from the group consisting of preparations serving for nutrition, for oral care or for pleasure, semifinished products, odoriferous, aroma or flavor substance compositions or seasoning mixtures. Preferred preparations according to the invention are listed below: bakery products (for example bread, dry biscuits, cakes, muffins, waffles, baking mixes, other pastry products), confectionery (for example white, milk or dark chocolates, filled chocolates (for example filled with aromatized fondant mass of the After Eight type), chocolate bars, other bar products, chewing candies, fruit gums, hard-boiled and soft candies, chewing gum, sugar pearls, lollipops), capsules (preferably seamless capsules for direct consumption, preferably with a shell based on gelatin and/or alginate), fat masses (for example fillings for baked goods such as for example biscuit fillings, fatty chocolate fillings, fatty chocolate bar fillings), toppings, alcoholic or non-alcoholic beverages (for example coffee, tea, wine, wine-based beverages, beer, beer-based beverages, liqueurs, spirits, brandies, fruit-based carbonated beverages, isotonic beverages, soft drinks, nectars, fruit and vegetable juices, fruit or vegetable juice preparations), instant beverages or instant powders (for example instant cocoa beverages, instant tea beverages, instant coffee beverages, instant desserts in powder form such as custard powder or jelly), meat products (for example ham, fresh or cured sausage preparations, spiced or marinated fresh or cured meat products), eggs or egg products (for example dried egg powder), cereal products and/or nut products (for example breakfast cereals, cornflakes, oat flakes, muesli, muesli bars, trail mix, sweet popcorn, nut bars, fruit and nut bars, precooked ready rice products), dairy products (for example milk beverages, milk ice cream, yogurt, custard, kefir, curd cheese, soft cheese, hard cheese, dried milk powder, whey, butter, buttermilk, products containing partially or entirely hydrolyzed milk protein), products made from soya protein or other soya bean fractions (for example soya milk and products produced therefrom, preparations containing soya lecithin, fermented products such as tofu or tempeh or products produced therefrom, soya sauces), fruit preparations (for example jams, fruit ice cream, fruit sauces, fruit fillings), vegetable preparations (for example ketchup, sauces, dried vegetables, deep-frozen vegetables, precooked vegetables, pickled vegetables, preserved vegetables), snack articles (for example baked or fried potato chips or potato dough products, bread dough products, maize- or peanut-based extrudates), fat- and oil-based products or corresponding emulsions (for example mayonnaise, remoulade, dressings, seasoning preparations), other ready-to-serve meals and soups (for example dried soups, instant soups, precooked soups), spices, seasoning mixtures and in particular powdered seasonings, which are for example used in snack food applications.

The present invention also relates to a method for enhancing the sweet flavor or the initial sweetness of a sweet-tasting substance or the sweet odor impression or the initial sweetness of an aroma substance which gives rise to a sweet odor impression, comprising the following step:
Mixing one or more sweet-tasting substances (component (b), as defined above) or one or more aroma substances which give rise to a sweet odor impression (component (c), as defined above) with a total quantity of an aroma composition according to the invention (component (a), as defined above), wherein the total quantity of aroma composition according to the invention (component (a)) in the preparation is sufficient to organoleptically enhance, preferably superproportionally, the sweet flavor impression of the sweet-tasting substance(s) (b) or the sweet odor impression of the aroma substance(s) (c) which give rise to a sweet odor impression, and in particular the initial sweetness.

One, two or in particular all of the components (a), (b) and (c) are preferably used here in one of their preferred embodiments as defined above.

With regard to the preferred quantities of aroma compositions according to the invention, see above. A total concentration of at least 10 ppm and at most 100 ppm in a ready-to-use preparation serving for oral care or a ready-to-consume preparation serving for nutrition or for pleasure is often particularly preferred here.

Overall, however, the explanations given above with regard to the aroma composition according to the invention, the use according to the invention and the preparation according to the invention also apply to the method according to the invention.

The present invention also relates to the use of one or more alkamides (i) selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);

to enhance the initial sweetness of a preparation comprising

(ii) hesperetin of the formula (I)

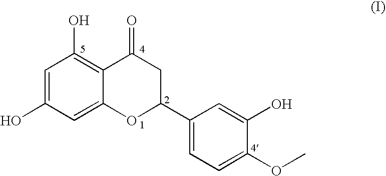

wherein the hesperetin of the formula (I) is present as the (2S)-enantiomer, (2R)-enantiomer or any desired mixture of the two enantiomers, and/or the salts thereof

and/or

(iii)
a 4-hydroxydihydrochalcone of the formula (II)

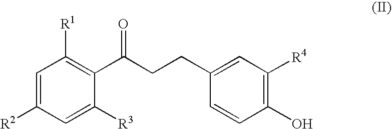

wherein

R¹, R², R³ and R⁴ in each case mutually independently denote H, OH or O-alkyl, with the proviso that at least one of the residues R¹, R² or R³ denotes OH,
a salt of such a 4-hydroxydihydrochalcone of the formula (II),
a mixture comprising or consisting of two or more different 4-hydroxydihydrochalcones of the formula (II), wherein R¹, R², R³ and R⁴ in each case have the meaning given above,
a mixture comprising or consisting of salts of two or more different 4-hydroxydihydrochalcones of the formula (II), wherein R¹, R², R³ and R⁴ in each case have the meaning given above
or
a mixture comprising or consisting of a 4-hydroxydihydrochalcone of the formula (II) or two or more different 4-hydroxydihydrochalcones of the formula (II), wherein R¹, R², R³ and R⁴ in each case have the meaning given above, and a salt of a 4-hydroxydihydrochalcone of the formula (II) or two or more salts of different 4-hydroxydihydrochalcones of the formula (II), wherein R¹, R², R³ and R⁴ in each case have the meaning given above,

and comprising

(b) one or more sweet-tasting substances

and/or

(c) one or more aroma substances which give rise to a sweet odor impression.

### EXAMPLES

The Examples serve to illustrate the invention without thereby limiting it. Unless otherwise stated, all stated values relate to weight.

### Example of Application 1

### Aroma Compositions

| | Preparation (quantity used in wt. %) | | | | |
|---|---|---|---|---|---|
| Constituent | A | B | C | D | E |
| 10 wt. % pellitorine | 0.25% | | 0.25% | 0.25% | 0.5% |
| (for example in | | | | | |
| accordance with | | | | | |
| WO 2004/043960) | | | | | |
| in 1,2-propylene | | | | | |
| glycol/diethyl | | | | | |
| malonate | | | | | |
| (constituent (i)) | | | | | |
| Jambu oleoresin | | 0.1% | 0.1% | | |
| containing 30 wt. % | | | | | |
| spilanthol in 1,2- | | | | | |
| propylene glycol | | | | | |
| (Robertet) | | | | | |
| (constituent (i)) | | | | | |
| Hesperetin | 2.5% | 2.5% | 2.5% | 2.5% | 5% |
| (constituent (ii)) | | | | | |
| Phloretin | — | 2.5% | 2.5% | 2.5% | — |
| (compound 4) | | | | | |
| (constituent (iii)) | | | | | |
| Propylene glycol | to make | to make | to make | to make | — |
| | up to | up to | up to | up to 100 | |
| | 100 | 100 | 100 | | |
| Ethanol | — | — | — | — | to make |
| | | | | | up to 100 |

Constituents (i), (ii) and (iii) (substances or solutions) are mixed together in the above-stated proportions and then taken up with propylene glycol or ethanol and completely dissolved by gentle heating.

### Example of Application 2

### Spray-Dried Preparations as Semifinished Products for Aromatizing Finished Products

| | Preparation (quantity used in wt. %) | | | | |
|---|---|---|---|---|---|
| Constituent | A | B | C | D | E |
| Drinking water | 60.72% | 60.72% | 62.94% | 65.14% | 60.76% |
| Maltodextrin | 24.3% | 24.3% | 24.3% | 24.3% | 24.3% |
| from wheat | | | | | |
| Gum arabic | 6.1% | 6.1% | 6.1% | 6.1% | 6.1% |
| Pellitorine in | 0.08% | 0.08% | 0.06% | 0.06% | 0.04% |
| accordance | | | | | |
| with WO 2004/ | | | | | |
| 043960 | | | | | |
| (constituent (i)) | | | | | |
| Hesperetin | — | 4.4% | 4.4% | 2.2% | 8.8% |
| (constituent | | | | | |
| (ii)) | | | | | |
| Phloretin | 8.8% | 4.4% | 2.2% | 2.2% | — |
| (compound 4) | | | | | |
| (constituent | | | | | |
| (iii)) | | | | | |

The drinking water is introduced into a container and the maltodextrin and gum arabic are dissolved therein. Constituents (i), (ii) and (iii) are then emulsified into the carrier solution described above using a mixer (Turrax). The temperature of the resulting mixture should not exceed 30° C. The mixture is then spray-dried (setpoint temperature at inlet: 185-195° C., setpoint temperature at outlet: 70-75° C.). The spray-dried semifinished product contains approximately 18-22% of the aroma compositions according to the invention.

Spray-dried preparations can also be prepared with other aroma compositions according to the invention in a similar manner.

### Example of Application 3

### Combinations with Sweet-Tasting Substances as Sweeteners

| | Preparation (quantity used in wt. %) | | | | | | | |
|---|---|---|---|---|---|---|---|---|
| Constituent | A | B | C | D | E | F | G | H |
| Sucrose | 88 | 89 | 89 | 89 | 89 | | | 50 |
| Fructose | 10 | 10 | 10 | | | | | |
| Tagatose | | | | 10 | 10 | | | |
| High fructose corn syrup | | | | | | 99 | | |
| Maltitol | | | | | | | 99 | |
| Sorbitol | | | | | | | | 49 |
| Aroma composition A | 2 | | | | | 1 | | |
| according to Example of | | | | | | | | |
| application 1 | | | | | | | | |
| Aroma composition B | | 1 | | | | | | |
| according to Example of | | | | | | | | |
| application 1 | | | | | | | | |
| Aroma composition C | | | 1 | | | | | |
| according to Example of | | | | | | | | |
| application 1 | | | | | | | | |
| Aroma composition D | | | | 1 | | | | 1 |
| according to Example of | | | | | | | | |
| application 1 | | | | | | | | |
| Aroma composition E | | | | | 1 | | 1 | |
| according to Example of | | | | | | | | |
| application 1 | | | | | | | | |

The constituents are mixed together in the specified order; the aroma compositions are preferably introduced by spraying. The resulting product can be used as a sweetener for foodstuffs or products consumed for pleasure, for example coffee or tea.

As an example of the application, tea and the product are mixed together and packed into teabags made from filter paper. For use, pour 100 to 250 ml of boiling water onto a teabag and allow to infuse for 2 to 5 minutes.

### Example of Application 4

### Aroma Mixtures for Enhancing Sweetness

| | Preparation (quantity used in wt. %) | | | | | | | | | |
|---|---|---|---|---|---|---|---|---|---|---|
| Constituent | A | B | C | D | E | F | G | H | I | J |
| Vanilla aroma | 75.00 | | 75.00 | | | | | | | |
| (available from | | | | | | | | | | |
| Symrise for example) | | | | | | | | | | |
| Sugar aroma, black | | 2.00 | | | | | | | | |
| treacle type | | | | | | | | | | |
| Ethyl lactate | | | | 1.00 | 0.50 | 0.050 | | 0.50 | 1.00 | 0.05 |
| n-Propyl lactate | | | | 0.50 | | | | 0.50 | 0.50 | |
| n-Butyl lactate | | | | 0.30 | 0.30 | 0.030 | 1.80 | 0.30 | 0.30 | 0.03 |
| Diethyl malate | | | | 1.00 | | | 1.00 | 0.50 | 1.00 | |
| Diethyl tartrate | | | | 0.50 | | | | 0.50 | 0.50 | |
| Diethyl succinate | | | | 0.50 | | | | | 0.50 | 10.0 |
| Diethyl malonate | | | | 0.50 | | | 2.00 | | 0.50 | |
| Triethyl citrate | | | | 0.50 | | | | 0.50 | 0.50 | |
| Lactic acid | | | | 1.00 | 2.00 | 0.20 | 2.00 | | 2.00 | 0.20 |
| 10 wt. % pellitorine | 0.05 | 0.10 | 0.03 | 0.25 | 0.30 | 0.20 | 0.10 | 0.50 | 0.10 | 0.30 |
| (for example in | | | | | | | | | | |
| accordance with WO | | | | | | | | | | |
| 2004043960) in 1,2- | | | | | | | | | | |
| propylene | | | | | | | | | | |
| glycol/diethyl | | | | | | | | | | |
| malonate | | | | | | | | | | |
| (constituent (i)) | | | | | | | | | | |
| Hesperetin | | 2.45 | 0.30 | | 1.25 | | 1.25 | | 1.50 | 2.50 |
| (constituent (ii)) | | | | | | | | | | |
| Phloretin (compound | 0.625 | | 0.325 | 2.50 | 1.25 | 2.50 | 1.25 | 5.00 | 1.00 | 2.50 |
| 4) (constituent (iii)) | | | | | | | | | | |
| 1,2-Propylene glycol | to | to | to | to | to | to | to | to | to | to |
| | make | make | make | make | make | make | make | make | make | make |
| | up to | up to | up to | up to | up to | up to | up to | up to | up to | up to |
| | 100 | 100 | 100 | 100 | 100 | 100 | 100 | 100 | 100 | 100 |

The components specified in the table are mixed together in the specified order by stirring and optionally completely homogenized by heating to 20-50° C. Clear, mostly colorless or yellowish solutions are obtained which can be used as an aroma.

### Example of Application 5

### Soya Milk Beverage

Comparative preparation with sugar (A)

Comparative preparation with reduced sugar content and hesperetin (B)

Preparations according to the invention with reduced sugar content and aroma compositions according to the invention (C-E)

| | Preparation (stated values as wt. %) | | | | |
|---|---|---|---|---|---|
| Constituent | A | B | C | D | E |
| Sucrose | 6% | 4.8% | 4.8% | 4.8% | 4.8% |
| Hesperetin | — | 50 ppm | — | — | — |
| Aroma composition | — | — | 0.2% | — | 0.2% |
| A according to | | | | | |
| Example of | | | | | |
| application 1 | | | | | |
| Aroma composition | — | — | — | 0.2% | — |
| D according to | | | | | |
| Example of | | | | | |
| application 1 | | | | | |
| Cream aroma | — | — | — | — | 0.002% |
| (available from | | | | | |
| Symrise for | | | | | |
| example) | | | | | |
| Soya milk, Sojasun | make | make | make | make up | make up |
| brand, non- | up | up | up | to 100% | to 100% |
| aromatized and | to 100% | to 100% | to 100% | | |
| unsweetened | | | | | |

As compared with preparation A, the sweetness in preparation B was similar but the initial sweetness was too slight. In preparation C, D and E the overall sweetness and the initial sweetness were similar to preparation A.

### Example of Application 6

### Chewing Gums

### Example of Application 6a

| | | Quantity |
|---|---|---|
| | | used in |
| Part | Constituent | wt. % |
| A | Chewing gum base, company “Jagum T” | 30.00 |
| B | Powdered sorbitol | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% aqueous solution | 14.00 |
| | Glycerol | 1.00 |
| D | Spearmint aroma | 0.80 |
| | Aroma composition A from Example of application 1 | 0.20 |

Parts A to D are mixed together and vigorously kneaded. The crude mixture can be processed into ready-to-consume chewing gums, for example in the form of thin strips.

### Example of Application 6b

### Non-Stick Chewing Gum

Chewing gum base K1 consisted of 2.0% butyl rubber (isobutene/isoprene copolymer, MW 400,000), 6.0% polyisobutene (MW=43,800), 43.5% polyvinyl acetate (MW=12,000), 31.5% polyvinyl acetate (MW=47,000), 6.75% triacetine and 10.25% calcium carbonate. Chewing gum base K1 and the chewing gums can be produced in a similar manner to U.S. Pat. No. 5,601,858.

| | I (wt. %) | II (wt. %) | III (wt. %) |
|---|---|---|---|
| Chewing gum base K1 | 26.00 | 26.00 | 26.00 |
| Triacetine | 0.25 | 0.25 | 0.25 |
| Lecithin | 0.50 | 0.50 | 0.50 |
| Sorbitol, crystalline | 40.80 | 40.60 | 40.50 |
| Mannitol | 15.30 | 15.20 | 15.10 |
| Glycerol | 12.10 | 12.00 | 11.80 |
| Aspartame | 0.17 | 0.17 | 0.17 |
| Encapsulated aspartame | 1.08 | 1.08 | 1.08 |
| Amorphous silica | 1.00 | 1.00 | 1.00 |
| Cottonseed oil | 0.50 | 0.50 | 0.50 |
| Polyoxyethylene sorbitane | 1.00 | 1.00 | 1.00 |
| monolaurate (E 432) | | | |
| Encapsulated spearmint | 0.20 | 0.10 | 0.30 |
| aroma (contains L- | | | |
| carvone) | | | |
| Encapsulated wintergreen | — | 0.40 | — |
| aroma (contains methyl | | | |
| salicylate) | | | |
| Peppermint oil | 0.80 | 0.80 | 1.00 |
| Aroma composition D from | 0.20 | 0.40 | 0.50 |
| Example of application 1 | | | |
| L-Menthyl-L-lactate | 0.10 | — | 0.30 |

### Example of Application 6c

### Bubble Gum

The bubble gum can be produced in a similar manner to U.S. Pat. No. 5,093,136.

| | I (wt. %) | II (wt. %) |
|---|---|---|
| Styrene/butadiene copolymer | 19.50 | 17.50 |
| (SBR) | | |
| Polyisobutene | 8.00 | 8.00 |
| Powdered sorbitol | 49.19 | 47.19 |
| Sorbitol, 70%, in water | 9.20 | 22.20 |
| Hydrogenated starch hydrolysate | 9.00 | — |
| (HSH) | | |
| Glycerol | 3.00 | 2.00 |
| Aspartame | 0.10 | 0.10 |
| Encapsulated aspartame | 0.50 | 0.50 |
| Red and blue dye | 0.01 | 0.01 |
| Strawberry/raspberry aroma | 1.00 | 2.20 |
| Aroma composition A from | 0.50 | 0.30 |
| Example of application 1 | | |

The chewing gums of formulation (I) were shaped into compact balls, those of formulation (II) were shaped into hollow balls.

### Example of Application 6d

### Chewing Gum

Chewing gum base K2 consisted of 28.5% terpene resin, 33.9% polyvinyl acetate (MW=14,000), 16.25% hydrogenated vegetable oil, 5.5% mono- and diglycerides, 0.5% polyisobutene (MW 75,000), 2.0% butyl rubber (isobutene/isoprene copolymer), 4.6% amorphous silicon dioxide (water content approx. 2.5%), 0.05% antioxidant tert.-butylhydroxytoluene (BHT), 0.2% lecithin, and 8.5% calcium carbonate. Chewing gum base K2 and the chewing gums can be produced in a similar manner to U.S. Pat. No. 6,986,907.

| | I (wt. %) | II (wt. %) | III (wt. %) |
|---|---|---|---|
| Chewing gum base K2 | 25.30 | 27.30 | 26.30 |
| Sorbitol | 61.48 | 59.48 | 61.60 |
| Glycerol | 2.40 | 2.40 | 2.40 |
| Lecithin | 7.00 | 7.00 | 7.00 |
| Aspartame | 0.14 | 0.14 | 0.14 |
| Encapsulated aspartame | 0.68 | 0.68 | 0.48 |
| Menthol, spray-dried | 1.00 | 0.50 | 0.40 |
| Cherry aroma, spray-dried | — | 1.20 | — |
| Lemon aroma | 1.00 | 1.00 | 1.48 |
| Orange oil, natural | 0.80 | — | — |
| Aroma composition B from | 0.20 | | 0.20 |
| Example of application 1 | | | |
| Aroma composition A from | | 0.30 | |
| Example of application 1 | | | |

The chewing gums of formulations (I) and (II) were shaped into strips, those of formulation (III) were shaped into pellets.

### Example of Application 6e

### Caffeine-Containing Chewing Gum

| | | Quantity |
|---|---|---|
| | | used in |
| Part | Constituent | wt. % |
| A | Chewing gum base, company “Jagum T” | 30.70 |
| B | Powdered sorbitol | 37.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Caffeine | 4.00 |
| | Emulgum ® (Colloides Naturels, Inc.) | 0.30 |
| C | Sorbitol, 70% aqueous solution | 11.00 |
| | Glycerol | 1.00 |
| D | Spearmint aroma | 0.80 |
| | Aroma composition E from Example of application 2 | 0.50 |

Parts A to D are mixed together and vigorously kneaded. The crude mixture can be processed into ready-to-consume chewing gums, for example in the form of thin strips.

### Example of Application 7

### Toothpaste

| | | Quantity |
|---|---|---|
| | | used in |
| Part | Constituent | wt. % |
| A | Demineralized water | 22.00 |
| | Sorbitol, 70% aqueous solution | 45.00 |
| | Solbrol ® M, sodium salt (Bayer AG, p- | 0.15 |
| | hydroxybenzoic acid alkyl ester) | |
| | Trisodium phosphate | 0.10 |
| | Saccharin, 450x | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.53 |
| | Sodium lauryl sulfate | 1.50 |
| D | Peppermint aroma | 0.80 |
| | Aroma composition D from Example of application 1 | 0.20 |

The constituents of parts A and B are in each case individually premixed and thoroughly stirred together under a vacuum at 25-30° C. for 30 minutes. Part C is premixed and added to A and B; D is added and the mixture is thoroughly stirred under a vacuum at 25-30° C. for 30 minutes. After relieving the vacuum, the toothpaste is ready and can be packaged.

### Example of Application 8

### Reduced-Sugar Soft Drinks

Comparative preparation with normal sucrose content (A)

Comparative preparation with reduced sucrose content and hesperetin and phloretin (B)

Preparations according to the invention (C-H)

| | Preparation (quantity used in wt. %) | | | | | | | |
|---|---|---|---|---|---|---|---|---|
| Constituent | A | B | C | D | E | F | G | H |
| Water | 89.8 | 91.845 | 91.82 | 91.825 | 91.325 | 91.625 | 91.535 | 91.34 |
| Sucrose | 10.0 | 8.0 | 8.0 | 8.0 | 8.0 | 8.0 | 8.0 | 8.0 |
| Citric acid | 0.2 | 0.15 | 0.15 | 0.15 | 0.15 | 0.15 | 0.15 | 0.15 |
| Fructose | | | | | | 0.2 | | |
| Tagatose | | | | | | | 0.3 | |
| Maltitol syrup | | | | | 0.5 | | | |
| Erythritol | | | | | | | | 0.5 |
| Hesperetin | — | 0.003 | | | | | | |
| Phloretin | — | 0.002 | | | | | | |
| Aroma | — | — | 0.03 | — | — | — | 0.015 | — |
| composition C | | | | | | | | |
| from Example of | | | | | | | | |
| application 2 | | | | | | | | |
| Aroma | — | — | — | 0.025 | 0.025 | 0.025 | — | 0.01 |
| composition B | | | | | | | | |
| from Example of | | | | | | | | |
| application 2 | | | | | | | | |

The substances were prepared and topped up with water and dissolved. Preparations C to H according to the invention exhibited not only a similar overall sweetness to comparative preparations (A) and (B) but also a substantially improved profile progression, especially with regard to the initial sweetness.

### Example of Application 9

### Low-Fat Yogurts

Comparative preparation with sugar (A)

Preparations according to the invention with mixture of sweeteners and aroma compositions according to the invention (B-D)

| | Preparation (stated values as wt. %) | | | |
|---|---|---|---|---|
| Constituent | A | B | C | D |
| Sucrose | 10% | 8% | 7% | 6% |
| Tagatose | — | — | | 0.5% |
| Fructose | — | — | | 0.5% |
| Aroma composition | — | 0.05% | — | |
| A according to | | | | |
| Example of | | | | |
| application 2 | | | | |
| Aroma composition | — | — | 0.4% | — |
| A according to | | | | |
| Example of | | | | |
| application 1 | | | | |
| Aroma composition | — | — | — | 0.4% |
| D according to | | | | |
| Example of | | | | |
| application 1 | | | | |
| Yogurt, 0.1% fat | make up | make up | make up to | make up to |
| | to 100% | to 100% | 100% | 100% |

The constituents were mixed together and cooled at 5° C.

### Example of Application 10

### Use Together with Sweeteners in Low-Fat Yogurts

Comparative preparation with mixture of sweeteners (A)

Preparations according to the invention with mixture of sweeteners and aroma compositions according to the invention (B-D)

| | Preparation (stated values as wt. %) | | | |
|---|---|---|---|---|
| Constituent | A | B | C | D |
| D-Tagatose | 0.482% | 0.482% | 0.482% | 0.482% |
| Sucralose | 0.003% | 0.003% | 0.003% | 0.003% |
| Aspartame | 0.005% | 0.005% | 0.005% | 0.005% |
| Acesulfame K | 0.01% | 0.01% | 0.01% | 0.01% |
| Aroma composition | — | 0.05% | — | — |
| A according to | | | | |
| Example of | | | | |
| application 2 | | | | |
| Aroma composition | — | — | 0.4% | — |
| A according to | | | | |
| Example of | | | | |
| application 1 | | | | |
| Aroma composition | — | — | — | 0.4% |
| D according to | | | | |
| Example of | | | | |
| application 1 | | | | |
| Yogurt, 0.1% fat | make up | make up | make up to | make up to |
| | to 100% | to 100% | 100% | 100% |

The constituents were mixed together and cooled at 5° C.

### Example of Application 11

### Mixed Milk Beverage

Comparative preparation with sugar (A-B)

Preparations according to the invention with sugar and aroma compositions according to the invention (C-E)

| | Preparation | | | | |
|---|---|---|---|---|---|
| | (stated values as wt. %) | | | | |
| Constituent | A | B | C | D | E |
| Sucrose | 10.0 | 8.0 | 8.0 | 8.0 | 7.0 |
| Fructose | — | — | — | — | 0.5 |
| Tagatose | — | — | — | — | 0.5 |
| Aroma composition | — | — | 0.025 | — | — |
| A according to | | | | | |
| Example of | | | | | |
| application 2 | | | | | |
| Aroma composition | — | — | — | 0.2 | — |
| A according to | | | | | |
| Example of | | | | | |
| application 1 | | | | | |
| Aroma composition | — | — | — | — | 0.2 |
| D according to | | | | | |
| Example of | | | | | |
| application 1 | | | | | |

| | |
|---|---|
| UHT milk, 1.5% fat | make up to 100% |

The constituents were mixed together topped up with milk, stirred thoroughly, poured into bottles and stored in refrigerated conditions at 5° C.

### Example of Application 12

### Reduced-Sugar Tomato Ketchup

Comparative preparation with sugar (A)

Comparative preparation with reduced sugar content (B)

Preparations according to the invention with sugar and aroma compositions according to the invention (C-I)

| | Preparation (stated values as wt. %) | | | | | | | |
|---|---|---|---|---|---|---|---|---|
| Constituent | A | B | C | E | F | G | H | I |
| Common salt | 2 | 2 | 2 | 2 | 2 | 2 | 2 | 2 |
| Starch, Farinex | 1 | 1 | 1 | 1 | 1 | 1 | 1 | 1 |
| WM 55 | | | | | | | | |
| Sucrose | 12 | 9.6 | 9.2 | 8.4 | 9.6 | 9.6 | 8.4 | 8.4 |
| Double tomato | 40 | 40 | 40 | 40 | 30 | 30 | 30 | 30 |
| concentrate | | | | | | | | |
| Glucose syrup, 80 | 18 | 18 | 18 | 18 | 18 | 18 | 18 | 18 |
| Brix | | | | | | | | |
| Distilled vinegar | 7 | 7 | 7 | 7 | 3 | 3 | 3 | 3 |
| 10% | | | | | | | | |
| Water | 20 | 22.4 | 22.4 | 23.2 | 36.0 | 36.0 | 37.2 | 37.2 |
| Aroma | | | 0.4 | | | 0.4 | | |
| composition A | | | | | | | | |
| according to | | | | | | | | |
| Example of | | | | | | | | |
| application 1 | | | | | | | | |
| Aroma | | | | | | | 0.4 | |
| composition B | | | | | | | | |
| according to | | | | | | | | |
| Example of | | | | | | | | |
| application 1 | | | | | | | | |
| Aroma | | | | 0.4 | 0.4 | | | 0.4 |
| composition D | | | | | | | | |
| according to | | | | | | | | |
| Example of | | | | | | | | |
| application 1 | | | | | | | | |

The constituents are mixed together in the specified order and the ready-to-use ketchup is homogenized with the assistance of a stirrer, filled into bottles and sterilized.

### Example of Application 13

### Reduced-Sugar Ice Creams

Comparative preparation with sugar (A)

Comparative preparation with reduced sugar content (B)

Preparations according to the invention with sugar and aroma compositions according to the invention (C-F)

| | Preparation (content in wt. %) | | | | | |
|---|---|---|---|---|---|---|
| Constituent | A | B | C | D | E | F |
| Skimmed milk | 57.15 | 61.15 | 60.95 | 61.05 | 60.95 | 61.05 |
| Vegetable fat, melting range | 20.00 | 20.00 | 20.00 | 20.00 | 20.00 | 20.00 |
| 35-40° C. | | | | | | |
| Sugar (sucrose) | 12.00 | 8.00 | 8.00 | 8.00 | 8.00 | 8.00 |
| Skimmed milk powder | 5.00 | 5.00 | 5.00 | 5.00 | 5.00 | 5.00 |
| Glucose syrup (72% solids) | 5.00 | 5.00 | 5.00 | 5.00 | 5.00 | 5.00 |
| Emulsifier SE 30 (Grindstedt | 0.65 | 0.65 | 0.65 | 0.65 | 0.65 | 0.65 |
| Products, Denmark) | | | | | | |
| Aroma containing 0.1% | 0.20 | 0.20 | 0.20 | 0.20 | | |
| diacetyl and 1% vanillin | | | | | | |
| Aroma containing 0.1% | | | | | 0.20 | 0.20 |
| diacetyl trimer according to | | | | | | |
| WO 2006/058893, 0.1% | | | | | | |
| diacetyl and 1% vanillin | | | | | | |
| Aroma according to Example | | | 0.20 | | 0.20 | |
| of application 4, | | | | | | |
| preparation E | | | | | | |
| Aroma according to Example | | | | 0.10 | | 0.10 |
| of application 4, | | | | | | |
| preparation H | | | | | | |

The skimmed milk and glucose syrup were heated to 55° and sugar, skimmed milk powder and emulsifier added. The vegetable fat was preheated and the entire mass heated to 58° C. After addition of the aroma, the mixture was homogenized with the assistance of a continuous high-pressure homogenizer (180/50 bar). The mass obtained was tempered for 1 minute at 78° C., then cooled to 2-4° C. and incubated at this temperature for 10 h to mature. The matured mass was then packaged and stored in a frozen state at −18° C.

### Example of Application 14

### Ice Cream Suitable for Diabetics

An ice cream suitable for diabetics was produced from the following ingredients and packed into tubs in 95 ml portions:

Thickened skimmed milk, fructose syrup, strawberry pieces and strawberry puree (15%), vegetable fat, diet chocolate chips (3.5%, with soya lecithin as emulsifier), whey product, beetroot juice, locust bean gum, guar gum, carrageen, emulsifier (E 471), gelatin, citric acid as acidulant, strawberry aroma (containing 40 wt. % aroma composition A according to Example of application 1), relative to the total weight of strawberry aroma), carotene as coloring.

Nutritional value (per 95 ml):

Protein 1.8 g, carbohydrates 13.3 g (including fructose 9.5 g), fat 4.2 g.

### Example of Application 15

### Diet Chocolate Based on Maltitol

A chocolate suitable for diabetics was produced from the following ingredients and cast into square bars:

Maltitol, hazelnut mass, cocoa butter, skimmed milk powder, cocoa mass, inulin, concentrated butter, soya lecithins as emulsifier, vanilla aroma (containing vanilla pod extract, vanillin and 40 wt. % aroma composition D according to Example of application 1, relative to the total weight of the vanilla aroma).

Nutritional value (per 100 g):

Protein 8 g, carbohydrates 43 g (including maltitol 34 g), fat 34 g.

### Example of Application 16

### Diet Chocolate Based on Fructose

A chocolate suitable for diabetics was produced from the following ingredients and cast into square bars:

Cocoa mass, fructose, skimmed milk powder, cocoa butter, inulin, concentrated butter, soya lecithin as emulsifier, walnuts, table salt, vanilla aroma (containing vanillin and 40 wt. % aroma composition A according to Example of application 1, relative to the total weight of the vanilla aroma).

Nutritional value (per 100 g):

Protein 8.8 g, carbohydrates 34 g (including fructose 23 g, lactose 7.5 g, sucrose 1.4 g), fat 36 g; fiber 18.5 (including 12.2 g inulin); sodium: 0.10 g. Cocoa content at least 50 wt. %.

### Example of Application 17

### Reduced-Sugar Muesli Mix

| | | | B (according to |
|---|---|---|---|
| | | A | the invention; |
| | | (comparison) | reduced-sugar) |
| No. | | (wt. %) | (wt. %) |
| 1 | Oat flakes | 17.00 | 18.90 |
| 2 | Crispy oat flake clusters | 10.00 | 12.00 |
| 3 | Rice crispies | 16.90 | 17.80 |
| 4 | Cornflakes | 16.50 | 17.50 |
| 5 | Currents | 3.50 | 3.50 |
| 6 | Hazelnuts, chopped | 2.50 | 2.50 |
| 7 | Glucose syrup from wheat, DE 30 | 9.50 | 9.50 |
| 8 | Sucrose | 20.00 | 14.00 |
| 9 | Water | 4.00 | 4.00 |
| 10 | Citric acid powder, anhydrous | 0.10 | 0.10 |
| 11 | Aroma composition D according to | — | 0.20 |
| | Example of application 1 | | |

In each case, mix constituents 1 to 6 together in a rotary drum (mix 1). In each case, heat constituents 7 to 9 and add constituent 10 (and in formulation B also add constituent 11) (mix 2). Add mix 2 to mix 1 in each case and mix thoroughly. Lastly, pour the resulting muesli mix onto a baking tray and dry in an oven at 130° C. for 8 minutes.

### Example of Application 18

### Reduced-Sugar Fruit Gums

| | | B (according to |
|---|---|---|
| | | the invention; |
| | A (comparison) | reduced-sugar) |
| | (wt. %) | (wt. %) |
| Water | 23.70 | 25.60 |
| Sucrose | 34.50 | 8.20 |
| Glucose syrup, DE 40 | 31.89 | 30.09 |
| Iso Syrup C* Tru Sweet 01750 | 1.50 | 2.10 |
| (Cerestar GmbH) | | |
| Gelatin 240 Bloom | 8.20 | 9.40 |
| Polydextrose (Litesse ® Ultra, | — | 24.40 |
| Danisco Cultor GmbH) | | |
| Yellow and red coloring | 0.01 | 0.01 |
| Citric acid | 0.20 | |
| Aroma according to Example of | — | 0.20 |
| application 4, preparation F | | |

Polydextrose is an intrinsically non-sweet-tasting polysaccharide with a low calorific value.

### Example of Application 19

### Chocolate/Cappuccino Ice Cream

| | A (wt. %) | B (wt. %) |
|---|---|---|
| Glucose/fructose syrup | 14.10 | 14.10 |
| Sucrose | 10.00 | 7.50 |
| Skimmed milk powder | 5.00 | 5.00 |
| Cream (36% fat content) | 24.00 | 24.00 |
| Emulsifier and stabilizer | 0.50 | 0.50 |
| Cremodan ® 709VEG (Danisco) | | |
| Cocoa powder | 5.975 | 5.975 |
| Carrageenan | 0.025 | 0.025 |
| Water | 40.20 | 42.50 |
| Cappuccino aroma | 0.20 | 0.20 |
| Aroma composition A according | — | 0.20 |
| to Example of application 1 | | |

### Example of Application 20

### Gelatin Capsules for Direct Consumption

| | I (wt. %) | II (wt. %) | III (wt. %) |
|---|---|---|---|
| Gelatin shell: | | | |
| Glycerol | 2.014 | 2.014 | 2.014 |
| Gelatin 240 Bloom | 7.91 | 7.91 | 7.91 |
| Sucralose | 0.065 | 0.065 | 0.065 |
| Allura red | 0.006 | 0.006 | 0.006 |
| Brilliant blue | 0.005 | 0.005 | 0.005 |
| Core composition: | | | |
| Vegetable oil triglyceride | 79.49 | 68.55 | 58.55 |
| (coconut oil fraction) | | | |
| Orange aroma | 8.0 | 16.0 | 23.65 |
| Aroma composition A | 2.0 | 4.0 | 5.0 |
| according to Example of | | | |
| application 1 | | | |
| Neotame and aspartame | 0.01 | 0.05 | — |
| Sucralose | 0.10 | 0.15 | 0.40 |
| 2-Hydroxypropylmenthyl | 0.33 | 0.20 | — |
| carbonate | | | |
| 2-Hydroxyethylmenthyl | — | 0.20 | 1.00 |
| carbonate | | | |
| (1R,3R,4S) Menthyl-3- | — | 0.55 | 0.50 |
| carboxylic acid-N- | | | |
| ethylamide (WS-3) | | | |
| (−)-Menthone glycerol | — | 0.30 | 0.80 |
| acetal (Frescolat MGA) | | | |
| Vanillin | 0.07 | — | 0.10 |

The gelatin capsules suitable for direct consumption were produced in accordance with WO 2004/050069 and had a diameter of 5 mm, the weight ratio of core material to shell material being 90:10. The capsules opened in the mouth within less than 10 seconds and dissolved completely within less than 50 seconds.

### Further Embodiments

A first embodiment is an aroma composition consisting of or containing a substance (i) and a substance (ii) or a substance (iii) or a mixture of substances (ii) and (iii), wherein:

(i) consists of one or more alkamides selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);

(ii) is hesperetin of the formula (I)

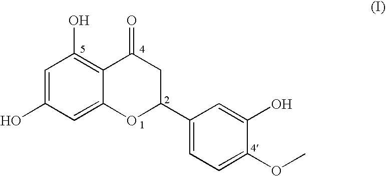

wherein the hesperetin of the formula (I) is present as the (2S)-enantiomer, (2R)-enantiomer or any desired mixture of the two enantiomers, and/or the salts thereof

and

(iii) is
a 4-hydroxydihydrochalcone of the formula (II)

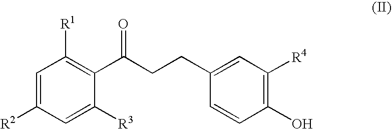

wherein

R¹, R², R³ and R⁴ in each case mutually independently denote H, OH or O-alkyl, with the proviso that at least one of the residues R¹, R² or R³ denotes OH,
a salt of such a 4-hydroxydihydrochalcone of the formula (II),
a mixture comprising or consisting of two or more different 4-hydroxydihydrochalcones of the formula (II), wherein R¹, R², R³ and R⁴ in each case have the meaning given above,
a mixture comprising or consisting of salts of two or more different 4-hydroxydihydrochalcones of the formula (II), wherein R¹, R², R³ and R⁴ in each case have the meaning given above
or
a mixture comprising or consisting of a 4-hydroxydihydrochalcone of the formula (II) or two or more different 4-hydroxydihydrochalcones of the formula (II), wherein R¹, R², R³ and R⁴ in each case have the meaning given above, and a salt of a 4-hydroxydihydrochalcone of the formula (II) or two or more salts of different 4-hydroxydihydrochalcones of the formula (II), wherein R¹, R², R³ and R⁴ in each case have the meaning given above.

A second embodiment is the aroma composition described in the first embodiment, wherein:

(i) consists of one or more alkamides selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-piperide (achilleamide), 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,4E,8Z,10E,12E-tetradecapentaenoic acid-N-(2-methylpropyl)amide (γ-sanshool) and 2E,4E,8Z,11Z-tetradecatetraenoic acid-N-(2-hydroxy-2-methylpropyl)amide (bungeanool);

and/or

(ii) is hesperetin of the formula (I)

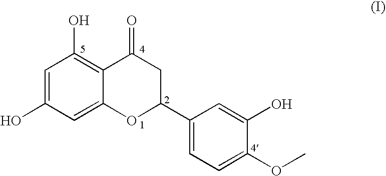

wherein the hesperetin of the formula (I) is present as the (2S)-enantiomer, (2R)-enantiomer or any desired mixture of the two enantiomers,

and/or

(iii) is a 4-hydroxydihydrochalcone of the formula (II), wherein in each case in the formula (II)

R¹ denotes OH

R² and R³, mutually independently, denote H or OH,

and

R⁴ denotes H or methoxy (OCH₃).

A third embodiment is the aroma composition as described in either one of the preceding preceding embodiments, wherein:

(i) consists of one or more alkamides selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,6Z,8E-decatrienoic acid-N-isobutylamide (spilanthol), 2E,6Z,8E,10E-dodecatetraenoic acid-N-(2-methylpropyl)amide (α-sanshool), 2E,4E,8Z,11Z-tetradecatetraenoic acid-N-(2-hydroxy-2-methylpropyl)amide (bungeanool);

and/or

(ii) is hesperetin of the formula (I)

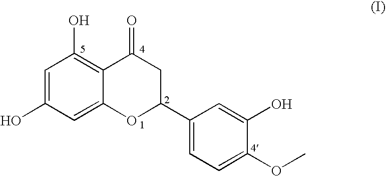

wherein the hesperetin of the formula (I) is present as the (2S)-enantiomer or any desired mixture of the two enantiomers,

and/or

(iii) consists of
a 4-hydroxydihydrochalcone of the formula (II) selected from the group consisting of:
3-(4-hydroxyphenyl)-1-(2-hydroxyphenyl)propan-1-one (2′,4-dihydroxydihydrochalcone; compound 1),
3-(4-hydroxyphenyl)-1-(2,4-dihydroxyphenyl)propan-1-one (2′,4,4′-trihydroxydihydrochalcone; davidigenin; compound 2),
3-(4-hydroxyphenyl)-1-(2,6-dihydroxyphenyl)propan-1-one (2′,4,6′-trihydroxydihydrochalcone; compound 3),
3-(4-hydroxyphenyl)-1-(2,4,6-trihydroxyphenyl)propan-1-one (2′,4,4′,6′-tetrahydroxydihydrochalcone; phloretin; compound 4),
3-(4-hydroxy-3-methoxyphenyl)-1-(2-hydroxyphenyl)propan-1-one (2′,4-dihydroxy-3-methoxydihydrochalcone; compound 5),
3-(4-hydroxy-3-methoxyphenyl)-1-(2,4-dihydroxyphenyl)propan-1-one (2′,4,4′-trihydroxy-3-methoxydihydrochalcone; compound 6),
3-(4-hydroxy-3-methoxyphenyl)-1-(2,6-dihydroxyphenyl)propan-1-one (2′,4,6′-trihydroxy-3-methoxydihydrochalcone; compound 7),
3-(4-hydroxy-3-methoxyphenyl)-1-(2,4,6-trihydroxyphenyl)propan-1-one (2′,4,4′,6′-tetrahydroxy-3-methoxydihydrochalcone; compound 8) and
3-(3,4-dihydroxyphenyl)-1-(2,4,6-trihydroxyphenyl)propan-1-one (2′,3,4,4′,6′-tetrahydroxydihydrochalcone; compound 9),
   a salt of such a 4-hydroxydihydrochalcone of the formula (II),
   a mixture comprising or consisting of two or more 4-hydroxydihydrochalcones of the formula (II) selected from said group,
   a mixture comprising or consisting of salts of two or more 4-hydroxydihydrochalcones of the formula (II) selected from said group
   or
   a mixture comprising or consisting of

one of the 4-hydroxydihydrochalcones of the formula (II) selected from said group or two or more 4-hydroxydihydrochalcones of the formula (II) selected from said group and

a salt of a 4-hydroxydihydrochalcone of the formula (II) selected from said group or two or more salts of different 4-hydroxydihydrochalcones of the formula (II) selected from said group.

A fourth embodiment is the aroma composition as described in any one of the preceding embodiments, wherein:

(i) consists of one or more alkamides selected from the group consisting of:

2E,4E-decadienoic acid-N-isobutylamide (pellitorine), 2E,6Z,8E-decatrienoic acid-N-isobutylamide (spilanthol), 2E,6Z,8E,10E-dodecatetraenoic acid-N-(2-methylpropyl)amide (α-sanshool) and 2E,4E,8Z,11Z-tetradecatetraenoic acid-N-(2-hydroxy-2-methylpropyl)amide (bungeanool);

and/or

(ii) is hesperetin of the formula (I)

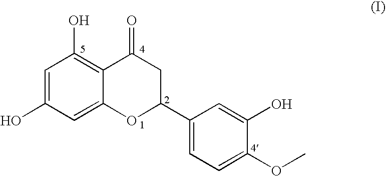

wherein the hesperetin of the formula (I) is present as the (2S)-enantiomer or any desired mixture of the two enantiomers,

and/or

(iii) consists of or comprises phloretin (compound 4).

A fifth embodiment is the aroma composition as described in any one of the preceding embodiments,wherein
the weight ratio of the total quantity of substances (i) to the total quantity of substances (ii) and (iii) is in a range from 1:1,000,000 to 1:1, preferably in a range from 1:10,000 to 1:10, particularly preferably in a range from 1:2000 to 1:50

and/or
the weight ratio of the total quantities of substances (ii) to substances (iii) is in the range from 1:10 to 10:1, particularly preferably in the range from 5:1 to 1:5 and especially preferably in the range from 7:3 to 3:7.

A sixth embodiment is the use of an aroma composition as described in any one of the preceding embodiments
to enhance the sweet flavor of a sweet-tasting substance or the sweet odor impression of an aroma substance which gives rise to a sweet odor impression.

A seventh embodiment is the use of an aroma composition as described in any one of the first through fifth embodiments to enhance the initial sweetness of a sweet-tasting substance or of an aroma substance which gives rise to a sweet odor impression.

An eighth embodiment is a preparation from the group consisting of preparations serving for nutrition, for oral care or for pleasure, semifinished products, odoriferous, aroma or flavor substance compositions or seasoning mixtures comprising the following components:

(a) an aroma composition as described in any one of the first through fifth embodiments

and additionally

(b) one or more sweet-tasting substances

and/or

(c) one or more aroma substances which give rise to a sweet odor impression,

wherein the total quantity of component (a) in the preparation is sufficient to organoleptically enhance the sweet flavor impression of the sweet-tasting substance(s) (b) or the sweet odor impression of the aroma substance(s) (c) which give rise to a sweet odor impression.

A ninth embodiment is the preparation as described in the eighth embodiment, comprising as component (b) one or more sugars, the total quantity of component (a) in the preparation being sufficient to impart, in comparison to a preparation or a semifinished product which, having an otherwise identical composition, contains no component (a) but at least 1.05 times the quantity of sugar, the same or an enhanced initial sweetness.

A tenth embodiment is the preparation as described in either the eighth or ninth embodiment, comprising

(b) one or more further sweet-tasting substances, wherein the further sweet-tasting substance(s) are selected from the group consisting of:
one or more carbohydrates selected from the group consisting of sucrose, trehalose, lactose, maltose, melizitose, melibiose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin and plant preparations containing one or more of the cited carbohydrates,
one or more sugar alcohols selected from the group consisting of glycerol, erythritol, threitol, arabitol, ribitol, xylitol, sorbitol, mannitol, maltitol, isomaltitol, dulcitol and lactitol,
one or more proteins and/or amino acids from the group consisting of miraculin, monellin, thaumatin, curculin, brazzein, glycine, D-leucine, D-threonine, D-asparagine, D-phenylalanine, D-tryptophan, L-proline,
one or more sweeteners from the group consisting of MAGAP, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatin and phyllodulcin,

and mixtures thereof

and/or

(c) one or more (optionally further) aroma substances which give rise to a sweet odor impression, wherein the further aroma substance(s) which give rise to a sweet odor impression are selected from the group consisting of:

vanillin, ethyl vanillin, ethyl vanillin isobutyrate (=3-ethoxy-4-isobutyryloxybenzaldehyde), Furaneol® (2,5-dimethyl-4-hydroxy-3(2H)-furanone) and derivatives (for example homofuraneol, 2-ethyl-4-hydroxy-5-methyl-3(2H)-furanone), homofuronol (2-ethyl-5-methyl-4-hydroxy-3(2 H )-furanone and 5-ethyl-2-methyl-4-hydroxy-3(2H)-furanone), maltol and derivatives (for example ethyl maltol), coumarin and derivatives, gamma-lactones (for example gamma-undecalactone, gamma-nonalactone), delta-lactones (for example 4-methyl delta-lactone, massoilactone, delta-decalactone, tuberolactone), methylsorbate, divanillin, 4-hydroxy-2(or 5)-ethyl-5(or 2)-methyl-3(2H)furanone, 2-hydroxy-3-methyl-2-cyclopentenones, 3-hydroxy-4,5-dimethyl-2(5H)-furanone, fruit esters and fruit lactones (for example acetic acid-n-butyl ester, acetic acid isoamyl ester, propionic acid ethyl ester, butyric acid ethyl ester, butyric acid-n-butyl ester, butyric acid isoamyl ester, 3-methyl butyric acid ethyl ester, n-hexanoic acid ethyl ester, n-hexanoic acid allyl ester,

n-hexanoic acid-n-butyl ester, n-octanoic acid ethyl ester, ethyl-3-methyl-3-phenyl glycidate, ethyl-2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenyl acetaldehyde.

An eleventh embodiment is the preparation serving for nutrition, for oral care or for pleasure as described in any one of the eighth through tenth embodiments, comprising a total quantity in the range from 0.1 to 150 ppm, preferably in the range from 1 to 50 ppm, particularly preferably in the range from 10 to 50 ppm, of an aroma composition as described in any one of the first through fourth embodiments, relative to the total weight of the preparation.

A twelfth embodiment is the preparation serving for nutrition, for oral care or for pleasure as described in any one of the eighth through eleventh embodiments, wherein the total quantity
of substances (i) is in the range from 0.005 to 5 ppm, preferably 0.02 to 2 ppm, particularly preferably 0.05 to 0.5 ppm,
of substances (ii) is in the range from 0.5 to 500 ppm, preferably 10 to 200 ppm, particularly preferably 20 to 100 ppm,
and/or
of substances (iii) is in the range from 0.5 to 500 ppm, preferably 5 to 100 ppm, particularly preferably 10 to 40 ppm,
   wherein the total quantity of all components (i), (ii) and (iii) is in the range from 0.5 to 500 ppm, preferably in the range from 5 to 200 ppm, particularly preferably in the range from 10 to 100 ppm.

A thirteenth embodiment is the preparation serving for nutrition, for oral care or for pleasure as described in any one of the eighth through twelfth embodiments, wherein the preparation is selected from the group consisting of:
(A) confectionery,
(B) alcoholic or non-alcoholic beverages or instant beverages,
(C) cereal products and/or nut products,
(D) dairy products,
(E) products made from soya protein or other soya bean fractions,
(F) fruit and/or vegetable preparations,
(G) fat- and oil-based products or emulsions thereof,
(H) oral care products.

A fourteenth embodiment is the preparation as described in any one of the eighth through thirteenth embodiments, selected from the group consisting of semifinished products, odoriferous, aroma or flavor substance compositions or seasoning mixtures, comprising a total quantity in the range from 0.0001 wt. % to 95 wt. %, preferably 0.001 wt. % to 80 wt. %, particularly preferably 0.001 wt. % to 50 wt. %, of an aroma composition as described in any one of the first through fifth embodiments, relative to the total weight of the preparation.

A fifteenth embodiment is the semifinished product as described in any one of the eighth through tenth or fourteenth embodiments, characterized in that it is spray-dried.

A sixteenth embodiment is the preparation as described in any one of the eighth through fifteenth embodiments, comprising

as additional component (d)

one or more esters selected from the group consisting of lactic acid-C₁-C₆-esters, tartaric acid di-C₁-C₄-esters, succinic acid di-C₁-C₄-esters, malonic acid di-C₁-C₄-esters, malic acid di-C₁-C₄-esters, citric acid di-C₁-C₄-esters and citric acid tri-C₁-C₄-esters,

and/or

one or more solvents selected from the group consisting of 1,2-propylene glycol, dimethyl sulfoxide, ethanol and ethanol/water blends.

A seventeenth embodiment is the preparation as described in any one of the eighth through sixteenth embodiments, further comprising at least one further substance for masking or reducing a bitter, metallic, chalky, acidic or astringent taste impression or for enhancing a sweet, salty or umami taste impression.

An eighteenth embodiment is a method for enhancing the sweet flavor or the initial sweetness of a sweet-tasting substance or the sweet odor impression or the initial sweetness of an aroma substance which gives rise to a sweet odor impression, comprising the following step:
Mixing one or more sweet-tasting substances (component (b)) or one or more aroma substances which give rise to a sweet odor impression (component (c)) with a total quantity of an aroma composition according to the invention (component (a)) as described in one of the first through fifth embodiments, wherein the total quantity of aroma composition according to the invention (component (a)) in the preparation is sufficient to organoleptically enhance the sweet flavor impression of the sweet-tasting substance(s) (b) or the sweet odor impression of the aroma substance(s) (c) which give rise to a sweet odor impression, and in particular the initial sweetness.

A nineteenth embodiment is the use of one or more alkamides (i) selected from the group consisting of:

2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);

to enhance the initial sweetness of a preparation comprising

(ii) hesperetin of the formula (I)

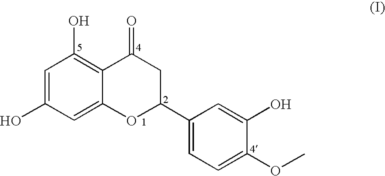

wherein the hesperetin of the formula (I) is present as the (2S)-enantiomer, (2R)-enantiomer or any desired mixture of the two enantiomers, and/or the salts thereof

and/or

(iii)
a 4-hydroxydihydrochalcone of the formula (II)

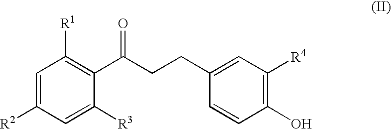

wherein

R¹, R², R³ and R⁴ in each case mutually independently denote H, OH or O-alkyl, with the proviso that at least one of the residues R¹, R² or R³ denotes OH,
a salt of such a 4-hydroxydihydrochalcone of the formula (II),
a mixture comprising or consisting of two or more different 4-hydroxydihydrochalcones of the formula (II), wherein R¹, R², R³ and R⁴ in each case have the meaning given above,
a mixture comprising or consisting of salts of two or more different 4-hydroxydihydrochalcones of the formula (11), wherein R¹, R², R³ and R⁴ in each case have the meaning given above
or
a mixture comprising or consisting of a 4-hydroxydihydrochalcone of the formula (II) or two or more different 4-hydroxydihydrochalcones of the formula (II), wherein R¹, R², R³ and R⁴ in each case have the meaning given above, and

a salt of a 4-hydroxydihydrochalcone of the formula (II) or two or more salts of different 4-hydroxydihydrochalcones of the formula (II), wherein R¹, R², R³ and R⁴ in each case have the meaning given above,

and comprising

(b) one or more sweet-tasting substances

and/or

(c) one or more aroma substances which give rise to a sweet odor impression.

1. An aroma composition comprising a substance (i) and either a substance (ii) or a substance (iii) or a mixture of substances (ii) and (iii), wherein:
(i) consists of one or more alkamides selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);
(ii) is hesperetin of the formula (I) 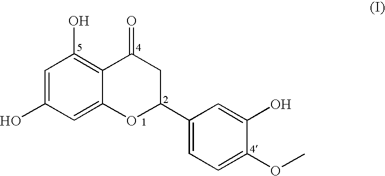
wherein the hesperetin of the formula (I) is present as the (2S)-enantiomer, (2R)-enantiomer or any desired mixture of the two enantiomers, and/or the salts thereof;
and
(iii) is
a 4-hydroxydihydrochalcone of the formula (II) 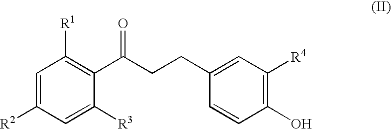
a salt of a 4-hydroxydihydrochalcone of the formula (II),
a mixture comprising two or more different 4-hydroxydihydrochalcones of the formula (II),
a mixture comprising salts of two or more different 4-hydroxydihydrochalcones of the formula (II),
or
a mixture comprising a 4-hydroxydihydrochalcone of the formula (II) or two or more different 4-hydroxydihydrochalcones of the formula (II), and a salt of a 4-hydroxydihydrochalcone of the formula (II) or two or more salts of different 4-hydroxydihydrochalcones of the formula (II),
wherein
R¹, R², R³ and R⁴ in each case independently are H, OH or O-alkyl, with the proviso that at least one of the residues R¹, R² or R³ is OH.

2. The aroma composition of claim 1, wherein:
(i) is one or more alkamides selected from the group consisting of
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-piperide (achilleamide), 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,4E,8Z,10E,12E-tetradecapentaenoic acid-N-(2-methylpropyl)amide (γ-sanshool) and 2E,4E,8Z,11Z-tetradecatetraenoic acid-N-(2-hydroxy-2-methylpropyl)amide (bungeanool); and optionally
(ii) is hesperetin of the formula (I) 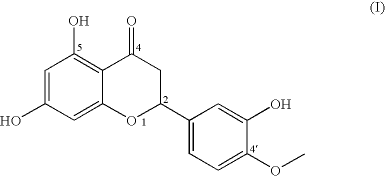
wherein the hesperetin of the formula (I) is present as the (2S)-enantiomer, (2R)-enantiomer or any desired mixture of the two enantiomers; and optionally
(iii) is a 4-hydroxydihydrochalcone of the formula (II), wherein
R¹ is OH
R² and R³, independently are H or OH,
and
R⁴ is H or methoxy (OCH₃).

3. The aroma composition of claim 1, wherein:
(i) is one or more alkamides selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,6Z,8E-decatrienoic acid-N-isobutylamide (spilanthol), 2E,6Z,8E,10E-dodecatetraenoic acid-N-(2-methylpropyl)amide (α-sanshool), 2E,4E,8Z,11Z-tetradecatetraenoic acid-N-(2-hydroxy-2-methylpropyl)amide (bungeanool); and optionally
(ii) is hesperetin of the formula (I) 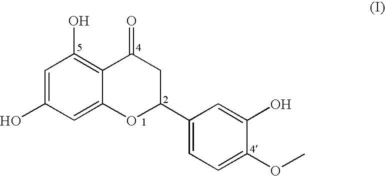
wherein the hesperetin of the formula (I) is present as the (2S)-enantiomer or any desired mixture of the two enantiomers; and optionally
(iii) is
a 4-hydroxydihydrochalcone of the formula (II) selected from the group consisting of:
3-(4-hydroxyphenyl)-1-(2-hydroxyphenyl)propan-1-one (2′,4-dihydroxydihydrochalcone; compound 1),
3-(4-hydroxyphenyl)-1-(2,4-dihydroxyphenyl)propan-1-one (2′,4,4′-trihydroxydihydrochalcone; davidigenin; compound 2),
3-(4-hydroxyphenyl)-1-(2,6-dihydroxyphenyl)propan-1-one (2′,4,6′-trihydroxydihydrochalcone; compound 3),
3-(4-hydroxyphenyl)-1-(2,4,6-trihydroxyphenyl)propan-1-one (2′,4,4′,6′-tetrahydroxydihydrochalcone; phloretin; compound 4),
3-(4-hydroxy-3-methoxyphenyl)-1-(2-hydroxyphenyl)propan-1-one (2′,4-dihydroxy-3-methoxydihydrochalcone; compound 5),
3-(4-hydroxy-3-methoxyphenyl)-1-(2,4-dihydroxyphenyl)propan-1-one (2′,4,4′-trihydroxy-3-methoxydihydrochalcone; compound 6),
3-(4-hydroxy-3-methoxyphenyl)-1-(2,6-dihydroxyphenyl)propan-1-one (2′,4,6′-trihydroxy-3-methoxydihydrochalcone; compound 7),
3-(4-hydroxy-3-methoxyphenyl)-1-(2,4,6-trihydroxyphenyl)propan-1-one (2′,4,4′,6′-tetrahydroxy-3-methoxydihydrochalcone; compound 8) and
3-(3,4-dihydroxyphenyl)-1-(2,4,6-trihydroxyphenyl)propan-1-one (2′,3,4,4′,6′-tetrahydroxydihydrochalcone; compound 9),
a salt of such a 4-hydroxydihydrochalcone of the formula (II),
a mixture comprising two or more of said 4-hydroxydihydrochalcones of the formula (II),
a mixture comprising salts of two or more of said 4-hydroxydihydrochalcones of the formula (II), or
a mixture comprising
one or more of said 4-hydroxydihydrochalcones of the formula (II) and
one or more salts of said 4-hydroxydihydrochalcone of the formula (II).

4. The aroma composition of claim 1, wherein:
(i) is one or more alkamides selected from the group consisting of:
2E,4E-decadienoic acid-N-isobutylamide (pellitorine), 2E,6Z,8E-decatrienoic acid-N-isobutylamide (spilanthol), 2E,6Z,8E,10E-dodecatetraenoic acid-N-(2-methylpropyl)amide (α-sanshool) and 2E,4E,8Z,11Z-tetradecatetraenoic acid-N-(2-hydroxy-2-methylpropyl)amide (bungeanool); and optionally
(ii) is hesperetin of the formula (I) 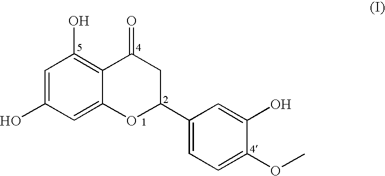
wherein the hesperetin of the formula (I) is present as the (2S)-enantiomer or any desired mixture of the two enantiomers, and optionally
(iii) is phloretin (compound 4).

5. The aroma composition of claim 1, wherein
the weight ratio of the total quantity of (i) to the total quantity of (ii) and (iii) is in a range from 1:1,000,000 to 1:1; and optionally
the weight ratio of the total quantities of (ii) to (iii) is in the range from 1:10 to 10:1.

6. A process to enhance the sweet flavor of a sweet-tasting substance or the sweet odor impression of an aroma substance, comprising
adding an aroma composition comprising a substance (i) and either a substance (ii) or a substance (iii) or a mixture of substances (ii) and (iii), wherein:
(i) consists of one or more alkamides selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);
(ii) is hesperetin of the formula (I) 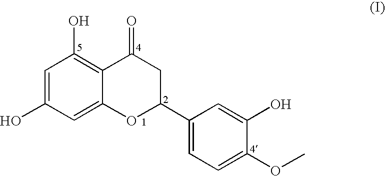
wherein the hesperetin of the formula (I) is present as the (2S)-enantiomer, (2R)-enantiomer or any desired mixture of the two enantiomers, and/or the salts thereof; and
(iii) is
a 4-hydroxydihydrochalcone of the formula (II) 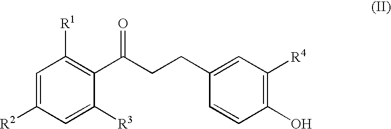
a salt of a 4-hydroxydihydrochalcone of the formula (II),
a mixture comprising two or more different 4-hydroxydihydrochalcones of the formula (II),
a mixture comprising salts of two or more different 4-hydroxydihydrochalcones of the formula (II),
or
a mixture comprising a 4-hydroxydihydrochalcone of the formula (II) or two or more different 4-hydroxydihydrochalcones of the formula (II), and a salt of a 4-hydroxydihydrochalcone of the formula (II) or two or more salts of different 4-hydroxydihydrochalcones of the formula (II),
wherein
R¹, R², R³ and R⁴ in each case independently are H, OH or O-alkyl, with the proviso that at least one of the residues R¹, R² or R³ is OH.
to enhance the sweet flavor of a sweet-tasting substance or the sweet odor impression of an aroma substance which gives rise to a sweet odor impression;
to a sweet-tasting substance or an aroma substance.

7. A process comprising
adding an aroma composition comprising a substance (i) and either a substance (ii) or a substance (iii) or a mixture of substances (ii) and (iii), wherein:
(i) consists of one or more alkamides selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);
(ii) is hesperetin of the formula (I) 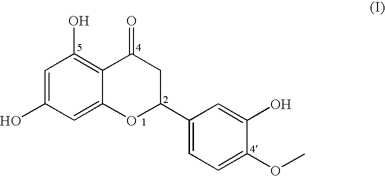
wherein the hesperetin of the formula (I) is present as the (2S)-enantiomer, (2R)-enantiomer or any desired mixture of the two enantiomers, and/or the salts thereof; and
(iii) is
a 4-hydroxydihydrochalcone of the formula (II) 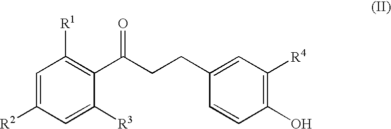
a salt of a 4-hydroxydihydrochalcone of the formula (II),
a mixture comprising two or more different 4-hydroxydihydrochalcones of the formula (II),
a mixture comprising salts of two or more different 4-hydroxydihydrochalcones of the formula (11),
or
a mixture comprising a 4-hydroxydihydrochalcone of the formula (II) or two or more different 4-hydroxydihydrochalcones of the formula (II), and a salt of a 4-hydroxydihydrochalcone of the formula (II) or two or more salts of different 4-hydroxydihydrochalcones of the formula (II),
wherein
R¹, R², R³ and R⁴ in each case independently are H, OH or O-alkyl, with the proviso that at least one of the residues R¹, R² or R³ is OH.
to enhance the sweet flavor of a sweet-tasting substance or the sweet odor impression of an aroma substance which gives rise to a sweet odor impression;
to a sweet-tasting substance or an aroma substance which gives rise to a sweet odor impression
enhancing the initial sweetness of the sweet-tasting substance or of the aroma substance.
The use of an aroma composition as claimed in any one of the preceding claims 1 to 5 to enhance the initial sweetness of a sweet-tasting substance or of an aroma substance which gives rise to a sweet odor impression.

8. A preparation for nutrition, for oral care or for pleasure, semifinished products, odoriferous, aroma or flavor substance compositions or seasoning mixtures comprising:
(a) an aroma composition comprising a substance (i) and either a substance (ii) or a substance (iii) or a mixture of substances (ii) and (iii), wherein:
(i) consists of one or more alkamides selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);
(ii) is hesperetin of the formula (I) 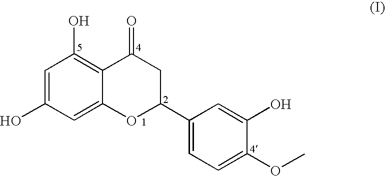
wherein the hesperetin of the formula (I) is present as the (2S)-enantiomer, (2R)-enantiomer or any desired mixture of the two enantiomers, and/or the salts thereof; and
(iii) is
a 4-hydroxydihydrochalcone of the formula (II) 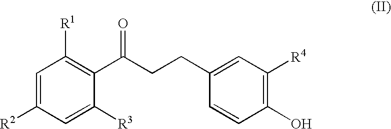
a salt of a 4-hydroxydihydrochalcone of the formula (II),
a mixture comprising two or more different 4-hydroxydihydrochalcones of the formula (II),
a mixture comprising salts of two or more different 4-hydroxydihydrochalcones of the formula (II),
or
a mixture comprising a 4-hydroxydihydrochalcone of the formula (II) or two or more different 4-hydroxydihydrochalcones of the formula (II), and a salt of a 4-hydroxydihydrochalcone of the formula (II) or two or more salts of different 4-hydroxydihydrochalcones of the formula (II),
wherein
R¹, R², R³ and R⁴ in each case independently are H, OH or O-alkyl, with the proviso that at least one of the residues R¹, R² or R³ is OH.
to enhance the sweet flavor of a sweet-tasting substance or the sweet odor impression of an aroma substance which gives rise to a sweet odor impression; and
(b) one or more sweet-tasting substances; and optionally
(c) one or more aroma substances which give rise to a sweet odor impression,
wherein the total quantity of component (a) in the preparation is sufficient to organoleptically enhance the sweet flavor impression of the sweet-tasting substance(s) (b) or the sweet odor impression of the aroma substance(s) (c).

9. The preparation of claim 8, wherein component (b) comprises one or more sugars; and the total quantity of component (a) in the preparation is sufficient to impart, in comparison to a preparation which, having an otherwise identical composition, contains no component (a) but at least 1.05 times the quantity of sugar, the same or an enhanced initial sweetness.

10. The preparation of claim 8, further comprising
(d) one or more further sweet-tasting substances, wherein the further sweet-tasting substance(s) are selected from the group consisting of:
one or more carbohydrates selected from the group consisting of sucrose, trehalose, lactose, maltose, melizitose, melibiose, raffinose, palatinose, lactulose, D-fructose, D-glucose, D-galactose, L-rhamnose, D-sorbose, D-mannose, D-tagatose, D-arabinose, L-arabinose, D-ribose, D-glyceraldehyde, maltodextrin and plant preparations containing one or more of the cited carbohydrates,
one or more sugar alcohols selected from the group consisting of glycerol, erythritol, threitol, arabitol, ribitol, xylitol, sorbitol, mannitol, maltitol, isomaltitol, dulcitol and lactitol,
one or more proteins and/or amino acids selected from the group consisting of miraculin, monellin, thaumatin, curculin, brazzein, glycine, D-leucine, D-threonine, D-asparagine, D-phenylalanine, D-tryptophan, L-proline,
one or more sweeteners selected from the group consisting of MAGAP, sodium cyclamate, acesulfame K, neohesperidin dihydrochalcone, saccharin sodium salt, aspartame, superaspartame, neotame, alitame, sucralose, stevioside, rebaudioside, lugduname, carrelame, sucrononate, sucrooctate, monatin and phyllodulcin,
and mixtures thereof; and optionally
(e) one or more further aroma substances which give rise to a sweet odor impression, selected from the group consisting of:
vanillin, ethyl vanillin, ethyl vanillin isobutyrate (=3-ethoxy-4-isobutyryloxybenzaldehyde), Furaneol® (2,5-dimethyl-4-hydroxy-3(2H)-furanone) and derivatives (for example homofuraneol, 2-ethyl-4-hydroxy-5-methyl-3(2H)-furanone), homofuronol (2-ethyl-5-methyl-4-hydroxy-3(2 H )-furanone and 5-ethyl-2-methyl-4-hydroxy-3(2H)-furanone), maltol and derivatives (for example ethyl maltol), coumarin and derivatives, gamma-lactones (for example gamma-undecalactone, gamma-nonalactone), delta-lactones (for example 4-methyl delta-lactone, massoilactone, delta-decalactone, tuberolactone), methylsorbate, divanillin, 4-hydroxy-2(or 5)-ethyl-5(or 2)-methyl-3(2H)furanone, 2-hydroxy-3-methyl-2-cyclopentenones, 3-hydroxy-4,5-dimethyl-2(5H)-furanone, fruit esters and fruit lactones (for example acetic acid-n-butyl ester, acetic acid isoamyl ester, propionic acid ethyl ester, butyric acid ethyl ester, butyric acid-n-butyl ester, butyric acid isoamyl ester, 3-methyl butyric acid ethyl ester, n-hexanoic acid ethyl ester, n-hexanoic acid allyl ester, n-hexanoic acid-n-butyl ester, n-octanoic acid ethyl ester, ethyl-3-methyl-3-phenyl glycidate, ethyl-2-trans-4-cis-decadienoate), 4-(p-hydroxyphenyl)-2-butanone, 1,1-dimethoxy-2,2,5-trimethyl-4-hexane, 2,6-dimethyl-5-hepten-1-al and phenyl acetaldehyde.

11. The preparation of claim 8, wherein component (a) is present in a total quantity of from 0.1 to 150 ppm, relative to the total weight of the preparation.

12. The preparation of claim 8, wherein
the total quantity of substances (i) is in the range from 0.005 to 5 ppm,
the total quantity of substances (ii) is in the range from 0.5 to 500 ppm, and optionally
the total quantity of substances (iii) is in the range from 0.5 to 500 ppm;
wherein the total quantity of all components (i), (ii) and (iii) is in the range from 0.5 to 500 ppm.

13. The preparation of claim 8, wherein the preparation is selected from the group consisting of:
(A) confectionery,
(B) alcoholic or non-alcoholic beverages or instant beverages,
(C) cereal products and/or nut products,
(D) dairy products,
(E) products made from soya protein or other soya bean fractions,
(F) fruit and/or vegetable preparations,
(G) fat- and oil-based products or emulsions thereof, and
(H) oral care products.

14. The preparation of claim 8, wherein the preparation is selected from the group consisting of semifinished products, odoriferous, aroma or flavor substance compositions or seasoning mixtures, and comprises a total quantity of component (a) in the range of from 0.0001 wt. % to 95 wt. %, relative to the total weight of the preparation.

15. The preparation of claim 8, wherein the preparation is spray-dried.

16. The preparation of claim 8, further comprising
as additional component (d)
one or more esters selected from the group consisting of lactic acid-C₁-C₆-esters, tartaric acid di-C₁-C₄-esters, succinic acid di-C₁-C₄-esters, malonic acid di-C₁-C₄-esters, malic acid di-C₁-C₄-esters, citric acid di-C₁-C₄-esters and citric acid tri-C₁-C₄-esters, and optionally
one or more solvents selected from the group consisting of 1,2-propylene glycol, dimethyl sulfoxide, ethanol and ethanol/water blends.

17. The preparation of claim 8, further comprising at least one further substance for masking or reducing a bitter, metallic, chalky, acidic or astringent taste impression or for enhancing a sweet, salty or umami taste impression.

18. A process comprising
adding one or more alkamides (i) selected from the group consisting of:
2E,4E-decadienoic acid-N-isobutylamide (pellitorine), 2E,4Z-decadienoic acid-N-isobutylamide (cis-pellitorine), 2Z,4Z-decadienoic acid-N-isobutylamide, 2Z,4E-decadienoic acid-N-isobutylamide, 2E,4E-decadienoic acid-N-([2S]-2-methylbutyl)amide, 2E,4E-decadienoic acid-N-([2S]-2-methylbutyl)amide, 2E,4E-decadienoic acid-N-([2R]-2-methylbutylamide), 2E,4Z-decadienoic acid-N-(2-methylbutyl)amide, 2E,4E-decadienoic acid-N-piperide (achilleamide), 2E,4E-decadienoic acid-N-piperide (sarmentine), 2E-decenoic acid-N-isobutylamide, 3E-decenoic acid-N-isobutylamide, 3E-nonenoic acid-N-isobutylamide, 2E,6Z,8E-decatrienoic acid-N-isobutylamide (spilanthol), 2E,6Z,8E-decatrienoic acid-N-([2S]-2-methylbutyl)amide (homospilanthol), 2E,6Z,8E-decatrienoic acid-N-([2R]-2-methylbutyl)amide, 2E-decen-4-ynoic acid-N-isobutylamide, 2Z-decen-4-ynoic acid-N-isobutylamide, 2E,6Z,8E,10E-dodecatetraenoic acid-N-(2-methylpropyl)amide (α-sanshool), 2E,6Z,8E,10E-dodecatetraenoic acid-N-(2-hydroxy-2-methylpropyl)amide (α-hydroxysanshool), 2E,6E,8E,10E-dodecatetraenoic acid-N-(2-hydroxy-2-methylpropyl)amide (β-hydroxysanshool), 2E,4E,8Z,10E,12E-tetradecapentaenoic acid-N-(2-hydroxy-2-methylpropyl)amide (γ-hydroxysanshool), 2E,4E,8E,10E,12E-tetradecapentaenoic acid-N-(2-hydroxy-2-methylpropyl)amide (γ-hydroxyisosanshool), 2E,4E,8Z,10E,12E-tetradecapentaenoic acid-N-(2-methyl-2-propenyl)amide (γ-dehydrosanshool), 2E,4E,8Z,10E,12E-tetradecapentaenoic acid-N-(2-methylpropyl)amide (γ-sanshool), 2E,4E,8Z,11Z-tetradecatetraenoic acid-N-(2-hydroxy-2-methylpropyl)amide (bungeanool), 2E,4E,8Z,11E-tetradecatetraenoic acid-N-(2-hydroxy-2-methylpropyl)amide (isobungeanool), 2E,4E,8Z-tetradecatrienoic acid-N-(2-hydroxy-2-methylpropyl)amide (dihydrobungeanool) and 2E,4E-tetradecadienoic acid-N-(2-hydroxy-2-methylpropyl)amide (tetrahydrobungeanool);
to a preparation comprising
(ii) hesperetin of the formula (I) 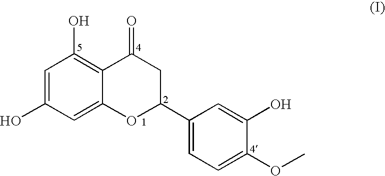
wherein the hesperetin of the formula (I) is present as the (2S)-enantiomer, (2R)-enantiomer or any desired mixture of the two enantiomers, and/or the salts thereof, and optionally
(iii)
a 4-hydroxydihydrochalcone of the formula (II) 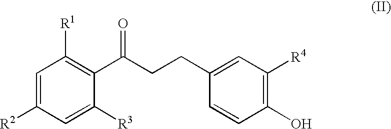
a salt of a 4-hydroxydihydrochalcone of the formula (II),
a mixture comprising two or more different 4-hydroxydihydrochalcones of the formula (II),
a mixture comprising salts of two or more different 4-hydroxydihydrochalcones of the formula (II), or
a mixture comprising one or more different 4-hydroxydihydrochalcones of the formula (II), and one ore more salts of different 4-hydroxydihydrochalcones of the formula (II),
wherein
R¹, R², R³ and R⁴ in each case independently are H, OH or O-alkyl, with the proviso that at least one of the residues R¹, R² or R³ is OH;
wherein said preparation further comprises one or more sweet-tasting substances, and optionally one or more aroma substances which give rise to a sweet odor impression.
